SEQUENCE LISTING (38 sequences)

SEQ ID NO: 1 - protein, 124 aa, synthetic construct
QFRELPDSVPQECTVRIYIVRGLELQPQDNNGLCDPYIKITLGKKVIEDRDHYIPNTLNPVFGRMYELSCYLPQEKDLKISVYDYDTFTRDEKVGETIIDLENRFLSRFGSHCGIPEEYCVSGV

SEQ ID NO: 2 - protein, 132 aa, synthetic construct
PFNITPRKAKKYYLRVIIWNTKDVILDEKSITGEEMSDIYVKGWVPGNEENKQKTDVHYRSLDGEGNFNWRFVFPFDYLPAEQLCIVAKKEHFWSIDQTEFRIPPRLIIQIWDNDKFSLDDYLGFLELDLRH

SEQ ID NO: 3 - protein, 119 aa, synthetic construct
PDLKAMNPLKAKTASLFEQKSMKGWWPCYAEKDGARVMAGKVEMTLEILNEKEADERPAGKGRDEPNMNPKLDLPNRPETSFLWFTNPCKTMKFIVWRRFKWVIIGLLFLLILLLFVAV

SEQ ID NO: 4 - protein, 136 aa, synthetic construct
MLRVIVESASNIPKTKFGKPDPIVSVIFKDEKKKTKKVDNELNPVWNEILEFDLRGIPLDFSSSLGIIVKDFETIGQNKLIGTATVALKDLTGDQSRSLPYKLISLLNERGQDTGATIDLVIGYDPPSAPHPNDLS

SEQ ID NO: 5 - protein, 58 aa, synthetic construct
LQTNIEALKSGIQGKIPANQLAELWLKLIDEVIEDTRYTLPLTEGKANVTVLDTQIRK

SEQ ID NO: 6 - protein, 72 aa, synthetic construct
WLDKLMQLTEEPQNSMPDIIIWMIRGEKRLAYARIPAHQVLYSTSGENASGKYCGKTQTIFLKYPQEKNNGP

SEQ ID NO: 7 - protein, 58 aa, synthetic construct
AVEKKFNSFAEGTFTVFAEMYENQALMFGKWGTSGLVGRHKFSDVTGKIKLKREFFLP

SEQ ID NO: 8 - protein, 57 aa, synthetic construct
DPERSLLTEADAGHTEFTDEVYQNESRYPGGDWKPAEDTYTDANGDKAASPSELTCP

SEQ ID NO: 9 - protein, 110 aa, synthetic construct
PQDFQIRVRVIEGRQLSGNNIRPVVKVHVCGQTHRTRIKRGNNPFFDELFFYNVNMTPSELMDEIISIRVYNSHSLRADCLMGEFKIDVGFVYDEPGHAVMRKWLLLNDP

SEQ ID NO: 10 - protein, 164 aa, synthetic construct
TFLLKIYRAEDIPQMDDAFSQTVKEIFGGNADKKNLVDPFVEVSFAGKKVCTNIIEKNANPEWNQVVNLQIKFPSVCEKIKLTIYDWDRLTKNDVVGTTYLHLSKIAASGGEVEDFSSSGTGAASYTVNTGETEVGFVPTFGPCYLNLYGSPREYTGFPDPYDE

SEQ ID NO: 11 - protein, 134 aa, synthetic construct
TPIVSCNFDRVYIYHLRCYVYQARNLLALDKDSFSDPYAHICFLHRSKTTEIIHSTLNPTWDQTIIFDEVEIYGEPQTVLQNPPKVIMELFDNDQVGKDEFLGRSIFSPVVKLNSEMDITPKLLWHPVMNGDKA

SEQ ID NO: 12 - protein, 77 aa, synthetic construct
RNMKNFQMASITSPSLVVECGGERVESVVIKNLKKTPNFPSSVLFMKVFLPKEELYMPPLVIKVIDHRQFGRKPVVG

SEQ ID NO: 13 - DNA, 372 bp, synthetic construct
  source 1..372: mol_type = other DNA
cagtttcgggaattacctgacagcgtcccacaggaatgcacggttaggatttacattgttcgaggcttagagctccagccccaggacaacaatggcctgtgtgacccttacataaaaataacactgggcaaaaaagtcattgaagaccgagatcactacattcccaacactctcaacccagtctttggcaggatgtacgaactgagctgctacttacctcaagaaaaagacctgaaaatttctgtctatgattatgacacctttacccgggatgaaaaagtaggagagacaattattgatctggaaaaccgattcctttcccgctttgggtcccactgcggcataccagaggagtactgtgtttctggagtc

SEQ ID NO: 14 - DNA, 396 bp, synthetic construct
  source 1..396: mol_type = other DNA
cctttcaacatcacaccccggaaagccaagaaatactacctgcgtgtgatcatctggaacaccaaggatgttatcttggatgagaaaagcatcacaggagaggaaatgagtgacatctacgtcaaaggctgggttcctggcaatgaagaaaacaaacagaaaacagatgtccattacagatctttggatggtgaagggaattttaactggcgatttgttttcccgtttgactaccttccagccgaacaactctgtatcgttgcgaaaaaagagcatttctggagtattgaccaaacggaatttcgaatcccacccaggctgatcattcagatatgggacaatgacaagttttctctggatgactacttgggtttcctagaacttgacttgcgtcac

SEQ ID NO: 15 - DNA, 357 bp, synthetic construct
  source 1..357: mol_type = other DNA
ccggacctcaaagccatgaacccccttaaagccaagacagcctccctctttgagcagaagtccatgaaaggatggtggccatgctacgcagagaaagatggcgcccgcgtaatggctgggaaagtggagatgacattggaaatcctcaacgagaaggaggccgacgagaggccagccgggaaggggcgggacgaacccaacatgaaccccaagctggacttaccaaatcgaccagaaacctccttcctctggttcaccaacccatgcaagaccatgaagttcatcgtgtggcgccgctttaagtgggtcatcatcggcttgctgttcctgcttatcctgctgctcttcgtggccgtg

SEQ ID NO: 16 - DNA, 408 bp, synthetic construct
  source 1..408: mol_type = other DNA
atgctgcgagtgattgtggaatctgccagcaatatccctaaaacgaaatttggcaagccggatcctattgtttctgtcatttttaaggatgagaaaaagaaaacaaagaaagttgataatgaattgaaccctgtctggaatgagattttggagtttgacttgaggggtataccactggacttttcatcttcccttgggattattgtgaaagattttgagacaattggacaaaataaattaattggcacggcgactgtagccctgaaggacctgactggtgaccagagcagatccctgccgtacaagctgatctccctgctaaatgaaagagggcaagatactggggccaccattgacttggtgatcggctatgatccgccttctgctccacatccaaatgacctgagc

SEQ ID NO: 17 - DNA, 174 bp, synthetic construct
  source 1..174: mol_type = other DNA
ctgcaaacaaatatagaagctctaaaatcagggatacaaggtaaaattcctgcaaaccagctggctgaattgtggctgaagctgatagatgaagttatagaagacacgagatacacgttgcctctcacagaaggaaaagccaacgtcacagttctcgatactcagatccgaaag

SEQ ID NO: 18 - DNA, 216 bp, synthetic construct
  source 1..216: mol_type = other DNA
tggcttgataaattaatgcagctgactgaagagccacagaacagcatgcctgacatcatcatctggatgatccggggagagaagagactggcctatgcacgaattcccgcacatcaggtcttgtactccaccagtggtgagaatgcatctggaaaatactgtgggaaaacccaaaccatctttctgaagtatccacaggagaaaaacaacgggcca

SEQ ID NO: 19 - DNA, 174 bp, synthetic construct
  source 1..174: mol_type = other DNA
gctgtggagaagaagtttaacagcttcgcagaaggaactttcaccgtctttgctgaaatgtatgaaaatcaagctctcatgtttggaaaatggggtacttctggattagtaggacgtcataagttttctgatgtcacaggaaaaataaaactcaagagggaattttttctgcct

SEQ ID NO: 20 - DNA, 171 bp, synthetic construct
  source 1..171: mol_type = other DNA
gatcctgaaagaagcttgctgactgaggcagatgcaggtcacacggagttcactgatgaagtctaccagaacgagagccgctaccccgggggcgactggaagccggccgaggacacctacacggatgcgaacggcgataaagcagcatcacccagcgagttgacttgtcct

SEQ ID NO: 21 - DNA, 330 bp, synthetic construct
  source 1..330: mol_type = other DNA
ccacaggacttccagatccgcgtccgagtgattgagggccgacagttaagtggcaacaacataaggcctgtggtcaaagttcacgtctgtggccagacacaccgaacaagaatcaagagaggaaacaaccctttttttgatgagttgtttttctacaatgtcaacatgaccccttctgaattgatggatgagatcatcagcatccgggtttataattctcactctctgcgggcagattgtctgatgggggaatttaagattgatgttggatttgtttatgatgaacctggccatgctgtcatgagaaagtggcttcttctcaatgacccg

SEQ ID NO: 22 - DNA, 492 bp, synthetic construct
  source 1..492: mol_type = other DNA
accttcttgctgaaaatctaccgagctgaggacatcccccagatggatgatgccttctcacagacagtaaaggaaatatttggaggcaatgcagataagaaaaatctcgtggatccttttgtagaagtttcctttgctggaaaaaaggtttgtacaaacataattgagaagaatgcaaacccagagtggaatcaggtcgtcaatcttcagatcaagtttccttcagtgtgtgaaaaaataaaactaacaatatatgactgggaccgtcttactaaaaatgatgtagttggaacaacatatctacacctctctaaaattgctgcctctggtggggaagtggaagatttctcatcttcgggaactggggctgcatcatatacagtaaacacaggagaaacagaggtaggctttgttccaacgtttggaccttgttacctgaatctttatggaagccccagagagtacacgggattcccagacccctatgatgag

SEQ ID NO: 23 - DNA, 402 bp, synthetic construct
  source 1..402: mol_type = other DNA
acccccattgtttcctgcaattttgacagagtctacatctaccatctgcgctgctatgtctatcaagccagaaacctcttggctttagataaggatagcttttcagatccatatgctcatatctgtttcctccatcggagcaaaaccactgagatcatccattcaaccctgaatcccacgtgggaccaaacaattatattcgatgaagttgaaatctatggggaaccccaaacagttctacagaatccacccaaagttatcatggaactttttgacaatgaccaagtgggcaaagatgaatttttaggacgaagcattttctctcctgtggtgaaactgaactcagaaatggacatcacacccaaacttctctggcacccagtaatgaatggagacaaagcc

SEQ ID NO: 24 - DNA, 231 bp, synthetic construct
  source 1..231: mol_type = other DNA
agaaatatgaaaaacttccagatggcttctatcacatcccccagtcttgttgtggagtgtggaggagaaagggtggaatcggtggtgatcaaaaaccttaagaagacacccaactttccaagttctgttctcttcatgaaagtgttcttgcccaaggaggaattgtacatgcccccactggtgatcaaggtcatcgaccacaggcagtttgggcggaagcctgtcgtcggc

SEQ ID NO: 25 - protein, 537 aa, synthetic construct
MVPAPPRQFRELPDSVPQECTVRIYIVRGLELQPQDNNGLCDPYIKITLGKKVIEDRDHYIPNTLNPVFGRMYELSCYLPQEKDLKISVYDYDTFTRDEKVGETIIDLENRFLSRFGSHCGIPEEYCVSGVNTWRDQLRPTQLLQNVARFKGFPQPILSEDGSRIRYGGRDYSLDEFEANKILHQHLGAPEERLALHILRTQGLVPEHVETRTLHSTFQPNISQGKLQMWVDVFPKSLGPPGPPFNITPRKAKKYYLRVIIWNTKDVILDEKSITGEEMSDIYVKGWVPGNEENKQKTDVHYRSLDGEGNFNWRFVFPFDYLPAEQLCIVAKKEHFWSIDQTEFRIPPRLIIQIWDNDKFSLDDYLGFLELDLRHTIIPAKSPEKCRLDMIPDLKAMNPLKAKTASLFEQKSMKGWWPCYAEKDGARVMAGKVEMTLEILNEKEADERPAGKGRDEPNMNPKLDLPNRPETSFLWFTNPCKTMKFIVWRRFKWVIIGLLFLLILLLFVAVLLYSLPNYLSMKIVKPNVYPYDVPDYA

SEQ ID NO: 26 - protein, 386 aa, synthetic construct
MATTMGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRAASRASSRPRPDDLEI

SEQ ID NO: 27 - DNA, 3351 bp, synthetic construct
  source 1..3351: mol_type = other DNA
atggtgccagcccctcccagacagtttcgggaattacctgacagcgtcccacaggaatgcacggttaggatttacattgttcgaggcttagagctccagccccaggacaacaatggcctgtgtgacccttacataaaaataacactgggcaaaaaagtcattgaagaccgagatcactacattcccaacactctcaacccagtctttggcaggatgtacgaactgagctgctacttacctcaagaaaaagacctgaaaatttctgtctatgattatgacacctttacccgggatgaaaaagtaggagagacaattattgatctggaaaaccgattcctttcccgctttgggtcccactgcggcataccagaggagtactgtgtttctggagtcaatacctggcgagatcaactgagaccaacacagctgcttcaaaatgtcgccagattcaaaggcttcccacaacccatcctttccgaagatgggagtagaatcagatatggaggacgagactacagcttggatgaatttgaagccaacaaaatcctgcaccagcacctcggggcccctgaagagcggcttgctcttcacatcctcaggactcaggggctggtccctgagcacgtggaaacaaggactttgcacagcaccttccagcccaacatttcccagggaaaacttcagatgtgggtggatgttttccccaagagtttggggccaccaggccctcctttcaacatcacaccccggaaagccaagaaatactacctgcgtgtgatcatctggaacaccaaggatgttatcttggatgagaaaagcatcacaggagaggaaatgagtgacatctacgtcaaaggctgggttcctggcaatgaagaaaacaaacagaaaacagatgtccattacagatctttggatggtgaagggaattttaactggcgatttgttttcccgtttgactaccttccagccgaacaactctgtatcgttgcgaaaaaagagcatttctggagtattgaccaaacggaatttcgaatcccacccaggctgatcattcagatatgggacaatgacaagttttctctggatgactacttgggtttcctagaacttgacttgcgtcacacgatcattcctgcaaaatcaccagagaaatgcaggttggacatgattccggacctcaaagccatgaacccccttaaagccaagacagcctccctctttgagcagaagtccatgaaaggatggtggccatgctacgcagagaaagatggcgcccgcgtaatggctgggaaagtggagatgacattggaaatcctcaacgagaaggaggccgacgagaggccagccgggaaggggcgggacgaacccaacatgaaccccaagctggacttaccaaatcgaccagaaacctccttcctctggttcaccaacccatgcaagaccatgaagttcatcgtgtggcgccgctttaagtgggtcatcatcggcttgctgttcctgcttatcctgctgctcttcgtggccgtgctcctctactctttgccgaactatttgtcaatgaagattgtaaagccaaatgtgtacccatacgacgtcccagactacgcttaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattcaaaaaaactagctgctggacatgaattacagccactagccattgtggaccagcgaccttcaagcagagccgccagtcgtgccagcagcagacctcggcctgatgacctggagatctga

SEQ ID NO: 28 - DNA, 1029 bp, synthetic construct
  source 1..1029: mol_type = other DNA
atggcggtggaaggaggaatgaaatgtgtgaagttcttgctctacgtcctcctgctggccttttgcgcctgtgcagtgggactgattgccgtgggtgtcggggcacagcttgtcctgagtcagaccataatccagggggctacccctggctctctgttgccagtggtcatcatcgcagtgggtgtcttcctcttcctggtggcttttgtgggctgctgcggggcctgcaaggagaactattgtcttatgatcacgtttgccatctttctgtctcttatcatgttggtggaggtggccgcagccattgctggctatgtgtttagagataaggtgatgtcagagtttaataacaacttccggcagcagatggagaattacccgaaaaacaaccacactgcttcgatcctggacaggatgcaggcagattttaagtgctgtggggctgctaactacacagattgggagaaaatcccttccatgtcgaagaaccgagtccccgactcctgctgcattaatgttactgtgggctgtgggattaatttcaacgagaaggcgatccataaggagggctgtgtggagaagattgggggctggctgaggaaaaatgtgctggtggtagctgcagcagcccttggaattgcttttgtcgaggttttgggaattgtctttgcctgctgcctcgtgaagagtatcagaagtggctacgaggtgatggaattcggcggaggcgggtccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaa

SEQ ID NO: 29 - protein, 343 aa, synthetic construct
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVMEFGGGGSMAVPETRPNHTIYINNLNEKIKKDELKKSLYAIFSQFGQILDILVSRSLKMRGQAFVIFKEVSSATNALRSMQGFPFYDKPMRIQYAKTDSDIIAKMK

SEQ ID NO: 30 - DNA, 1056 bp, synthetic construct
  source 1..1056: mol_type = other DNA
atgtccggactcagatctcgagctcaagcttccggagtggagggctgcaccaagtgcatcaagtacctgctcttcgtcttcaatttcgtcttctggctggctggaggcgtgatcctgggtgtggccctgtggctccgccatgacccgcagaccaccaacctcctgtatctggagctgggagacaagcccgcgcccaacaccttctatgtaggcatctacatcctcatcgctgtgggcgctgtcatgatgttcgttggcttcctgggctgctacggggccatccaggaatcccagtgcctgctggggacgttcttcacctgcctggtcatcctgtttgcctgtgaggtggccgccggcatctggggctttgtcaacaaggaccagatcgccaaggatgtgaagcagttctatgaccaggccctacagcaggccgtggtggatgatgacgccaacaacgccaaggctgtggtgaagaccttccacgagacgcttgactgctgtggctccagcacactgactgctttgaccacctcagtgctcaagaacaatttgtgtccctcgggcagcaacatcatcagcaacctcttcaaggaggactgccaccagaagatcgatgacctcttctccgggaagctgtacctcatcggcattgctgccatcgtggtcgctgtgatcatgatcttcgagatgatcctgagcatggtgctgtgctgtggcatccggaacagctccgtgtacgaattcggcggaggcgggtccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaataa

SEQ ID NO: 31 - protein, 351 aa, synthetic construct
MSGLRSRAQASGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVYEFGGGGSMAVPETRPNHTIYINNLNEKIKKDELKKSLYAIFSQFGQILDILVSRSLKMRGQAFVIFKEVSSATNALRSMQGFPFYDKPMRIQYAKTDSDIIAKMK

SEQ ID NO: 32 - DNA, 2955 bp, synthetic construct
  source 1..2955: mol_type = other DNA
atggggcgcctggcctccaggccgctgctgctggcgctcctgtcgttggctctttgccgagggcgtgtggtgagagtccccacagcgaccctggttcgagtggtgggcactgagctggtcatcccctgcaacgtcagtgactatgatggccccagcgagcaaaactttgactggagcttctcatctttggggagcagctttgtggagcttgcaagcacctgggaggtggggttcccagcccagctgtaccaggagcggctgcagaggggcgagatcctgttaaggcggactgccaacgacgccgtggagctccacataaagaacgtccagccttcagaccaaggccactacaaatgttcaacccccagcacagatgccactgtccagggaaactatgaggacacagtgcaggttaaagtgctggccgactccctgcacgtgggccccagcgcgcggcccccgccgagcctgagcctgcgggagggggagcccttcgagctgcgctgcaccgccgcctccgcctcgccgctgcacacgcacctggcgctgctgtgggaggtgcaccgcggcccggccaggcggagcgtcctcgccctgacccacgagggcaggttccacccgggcctggggtacgagcagcgctaccacagtggggacgtgcgcctcgacaccgtgggcagcgacgcctaccgcctctcagtgtcccgggctctgtctgccgaccagggctcctacaggtgtatcgtcagcgagtggatcgccgagcagggcaactggcaggaaatccaagaaaaggccgtggaagttgccaccgtggtgatccagccatcagttctgcgagcagctgtgcccaagaatgtgtctgtggctgaaggaaaggaactggacctgacctgtaacatcacaacagaccgagccgatgacgtccggcccgaggtgacgtggtccttcagcaggatgcctgacagcaccctacctggctcccgcgtgttggcgcggcttgaccgtgattccctggtgcacagctcgcctcatgttgctttgagtcatgtggatgcacgctcctaccatttactggttcgggatgttagcaaagaaaactctggctactattactgccacgtgtccctgtgggcacccggacacaacaggagctggcacaaagtggcagaggccgtgtcttccccagctggtgtgggtgtgacctggctagaaccagactaccaggtgtacctgaatgcttccaaggtccccgggtttgcggatgaccccacagagctggcatgccgggtggtggacacgaagagtggggaggcgaatgtccgattcacggtttcgtggtactacaggatgaaccggcgcagcgacaatgtggtgaccagcgagctgcttgcagtcatggacggggactggacgctaaaatatggagagaggagcaagcagcgggcccaggatggagactttattttttctaaggaacatacagacacgttcaatttccggatccaaaggactacagaggaagacagaggcaattattactgtgttgtgtctgcctggaccaaacagcggaacaacagctgggtgaaaagcaaggatgtcttctccaagcctgttaacatattttgggcattagaagattccgtgcttgtggtgaaggcgaggcagccaaagcctttctttgctgccggaaatacatttgagatgacttgcaaagtatcttccaagaatattaagtcgccacgctactctgttctcatcatggctgagaagcctgtcggcgacctctccagtcccaatgaaacgaagtacatcatctctctggaccaggattctgtggtgaagctggagaattggacagatgcatcacgggtggatggcgttgttttagaaaaagtgcaggaggatgagttccgctatcgaatgtaccagactcaggtctcagacgcagggctgtaccgctgcatggtgacagcctggtctcctgtcaggggcagcctttggcgagaagcagcaaccagtctctccaatcctattgagatagacttccaaacctcaggtcctatatttaatgcttctgtgcattcagacacaccatcagtaattcggggagatctgatcaaattgttctgtatcatcactgtcgagggagcagcactggatccagatgacatggcctttgatgtgtcctggtttgcggtgcactcttttggcctggacaaggctcctgtgctcctgtcttccctggatcggaagggcatcgtgaccacctcccggagggactggaagagcgacctcagcctggagcgcgtgagtgtgctggaattcttgctgcaagtgcatggctccgaggaccaggactttggcaactactactgttccgtgactccatgggtgaagtcaccaacaggttcctggcagaaggaggcagagatccactccaagcccgtttttataactgtgaagatggatgtgctgaacgccttcaagtatcccttgctgatcggcgtcggtctgtccacggtcatcgggctcctgtcctgtctcatcgggtactgcagctcccactggtgttgtaagaaggaggttcaggagacacggcgcgagcgccgcaggctcatgtcgatggagatggacgaattcggcggaggcgggtccatggcagttcccgagacccgccctaaccacactatttatatcaacaacctcaatgagaagatcaagaaggatgagctaaaaaagtccctgtacgccatcttctcccagtttggccagatcctggatatcctggtatcacggagcctgaagatgaggggccaggcctttgtcatcttcaaggaggtcagcagcgccaccaacgccctgcgctccatgcagggtttccctttctatgacaaacctatgcgtatccagtatgccaagaccgactcagatatcattgccaagatgaaatag

SEQ ID NO: 33 - protein, 984 aa, synthetic construct
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMDEFGGGGSMAVPETRPNHTIYINNLNEKIKKDELKKSLYAIFSQFGQILDILVSRSLKMRGQAFVIFKEVSSATNALRSMQGFPFYDKPMRIQYAKTDSDIIAKMK

SEQ ID NO: 34 - DNA, 20 bp, synthetic construct
  source 1..20: mol_type = other DNA
aatccattgcactccggatt

SEQ ID NO: 35 - DNA, 21 bp, synthetic construct
  source 1..21: mol_type = other DNA
aatccattgcactccggattt

SEQ ID NO: 36 - DNA, 23 bp, synthetic construct
  source 1..23: mol_type = other DNA
ctgcagatatccagcacagtggc

SEQ ID NO: 37 - DNA, 18 bp, synthetic construct
  source 1..18: mol_type = other DNA
gcgcagcgcgcgcagcgc

SEQ ID NO: 38 - DNA, 42 bp, synthetic construct
  source 1..42: mol_type = other DNA
cgcctcccgggttcaagcgattctcctgcctcagcctcccga

(19) United States
(12) Patent Application Publication
(10) Pub. No.: US 2026/0274911 A1
(43) Pub. Date: Sep. 17, 2026

(54) Modified Myoferlin Protein and Method of Use Thereof to Increase Nanoparticle Payload Release

(71) Applicant: Gene Company (Pty)Ltd., Kelvin Grove (AU)

(72) Inventor: Kevin Morris, Big Bear City, CA (US)

(21) Appl. No.: 19/675,563

(22) Filed: May 13, 2026

Related U.S. Application Data
(63) Continuation of application No. PCT/US24/55789, filed on Nov. 13, 2024.
(60) Provisional application No. 63/598,499, filed on Nov. 13, 2023.
(60) Provisional application No. 63/607,552, filed on Dec. 7, 2023.

(51) Int. Cl. C07K 14/47; A61K 38/00; A61K 45/06; A61K 47/69

(52) U.S. Cl. CPC C07K 14/4707; A61K 45/06; A61K 47/6929; C07K 14/4702; A61K 38/00; C07K 2319/03

(57) ABSTRACT

A modified myoferlin protein capable of being packaged into extracellular vesicles (EVs) to facilitate release of EV payloads into recipient cells. The modified myoferlin protein enhances the release of payload at the targeted site. The present invention further provides a modified myoferlin-encapsulating nanoparticle comprising an exosome encapsulating any embodiment of the modified myoferlin of the present invention and one or more cargo RNAs to enhance release of the cargo RNAs at the targeted site.

### CROSS REFERENCE TO RELATED APPLICATION

The present application is a national stage filing under 35 U.S.C. § 371 of international PCT application, PCT/US24/55789, filed Nov. 13, 2024, which claims priority under 35 U.S.C. § 119 (e) to U.S. provisional patent application, U.S. Ser. No. 63/607,552, filed Dec. 7, 2023 and U.S. provisional patent application, U.S. Ser. No. 63/598,499, filed Nov. 13, 2023, each of which is incorporated herein by reference.

### INCORPORATION BY REFERENCE OF A SEQUENCE LISTING XML

A Sequence Listing is provided herewith as a Sequence Listing XML, “Modified_Myoferlin.xml” created on Nov. 12, 2024 and having a size of 49,793 bytes. The contents of the Sequence Listing XML are incorporated by reference herein in their entirety.

### FIELD OF THE INVENTION

The present invention is related to a composition comprising a modified myoferlin protein and method of use thereof for increasing the release of payload from a nanoparticle.

### BACKGROUND OF THE INVENTION

Myoferlin (MYOF) is a protein that in humans is encoded by the MYOF gene and is a member of the Ferlin family of calcium-regulated vesicle fusion proteins and has been observed to facilitate cancer cell extracellular vesicle (EV) uptake into recipient cells (Blomme, 2016 #4760). Myoferlin contains C2 domains that play a role in calcium-mediated membrane fusion events, suggesting that it may be involved in membrane regeneration and repair. Myoferlin also contains a FerA domain which has been shown to interact with the membrane, suggesting that FerA domain in myoferlin may contribute to myoferlin's membrane interaction mechanism. The protein also contains domains involved in receptor-mediated trafficking, which may help to reduce trafficking to the lysosome and facilitate endosomal release of payloads from nanoparticles. As only 3-5% of mRNA payloads are released from lipid nanoparticles, there is a need to substantially increase release of payloads from nanoparticles using modified MYOF.

### SUMMARY OF THE INVENTION

The present invention provides a modified myoferlin protein comprising one or more C2 domains and one or more non-C2 domains wherein the one or more C2 domains comprises a C2A domain, a C2B domain, a C2C domain, a C2D domain, a C2E domain, a C2F domain, a C2G domain, or a combination thereof; wherein the one or more non-C2 domain comprise a transmembrane domain, a FerA domain, a FerB domain, a DysFN-1, domain, a DysFN-2 domain derived from a human myoferlin protein, or a combination thereof and wherein the modified myoferlin protein does not comprise one or more of C2B domain, C2C domain, C2D domain and/or C2E domains. In an embodiment, the modified myoferlin protein of the present invention comprises a polypeptide comprising a C2F domain, a C2G domain, a transmembrane domain, or a combination thereof. In an embodiment, the modified myoferlin protein of the present invention comprises a polypeptide comprising a C2A domain, a FerA domain, a FerB domain, a DysFN-1 domain, a DysFN-2 domain, a transmembrane domain, or a combination thereof.

### BRIEF DESCRIPTION OF THE DRAWINGS

FIGS. 1A and 1B illustrate effect of myoferlin (MYOF) on EV uptake relative to the EXOtic system. Using the EXOtic extracellular vesicle (EV) system [2] EVs were produced from HEK293 packaged with nanoluciferase mRNA and expression of this protein determined using a nanoluciferase assay in recipient cells. The HEK293T cell produced EVs were purified using a stepwise centrifugation protocol that ends with ultra-centrifugation and quantified using the Nanocyte 3000 Nanoparticle tracking analysis [3] to determine size and concentration. A total of 1×109 EVs per 10,000 HeLa cells were assessed for transfer of nanoluciferase activity at 24 hrs post-EV exposure.

FIG. 2 illustrates the schematics of (A) full length MYOF ~6.2 kB and (B)-(E) the various truncated MYOF constructs assessed in the present invention characterized for enhanced endosomal release of nano-luciferase from EVs.

FIG. 3 illustrates the effect of full-length and variant forms of MYOF on EV transfer of nanoluciferase.

FIG. 4 illustrates the plasmid map for pC2A-Dys-Fer myoferlin truncated variant.

FIG. 5 illustrates the plasmid map for pC2F-C2G myoferlin truncated variant.

FIG. 6 illustrates next generation dual expressing Myo/Cnx43 enhancer system. The truncated myoferlin pC2F-C2G MYOF determined previously, (FIG. 4), is expressed from the EF1a promoter and an internal IRES is used to express the downstream Connexion 43 with the S368A mutation. The various annotated domains found in the truncated Myoferlin are shown.

FIG. 7 illustrates the schematics for pC2F-C2G myoferlin truncated variant fused with Connexion 43 via IRES.

FIG. 8 illustrates the transient transwell assays used for the detection of enhanced exosome transfer.

FIG. 9 illustrates an embodiment of dual enhancer Myo/Cnx functional transfer of GFP mRNA payloads into recipient cells. Transient transwell assays were carried out with HEK293 producer cells transfected with various plasmids. The producer cells were grown in transwell culture with recipient HEK293 cells and 72 hrs later the culture RNAs collected and qRT-PCR carried out for GFP vs. beta actin and assess as recipient relative to producer. The P=value from a paired T-test is shown relative to the negative control treated cultures (E4) with standard deviations.

FIG. 10 illustrates the plasmid map for lentiviral vector p87_pVB240605-1047fgq_pLV-TrunMyo-IRES-Cnx43 used to generate stable exosome producing cell systems.

FIG. 11 illustrates the top candidate enhancers for shRNA exosome delivery. Transiently transfected HEK293 producer cells were generated with various plasmids and 24 hrs later grown in transwell plates with GFP reporter cells. The cells were collected 72 hrs later and assessed for repression of GFP relative to controls.

### DETAILED DESCRIPTION OF THE INVENTION

As used in this specification and in claims which follow, the singular forms “a”, “an” and “the” include plural referents unless the context clearly indicates otherwise. Thus, for example, reference to “an ingredient” includes mixtures of ingredients, reference to “an active pharmaceutical agent” includes more than one active pharmaceutical agent, and the like.

As used herein, the term “about” as a modifier to a quantity is intended to mean + or −20%, + or −15%, + or −10% or + or −5% inclusive of the quantity being modified. As used herein, the term “effective amount” or “a therapeutically effective amount” of a drug or pharmacologically active agent comprises administering an amount necessary to achieve a desired result. The exact amount required will vary from subject to subject, depending on the species, age, general condition of the subject, the severity of the disease, the particular active agent, its mode of administration, the desired outcome, and the like. In certain embodiments of the present invention, a “therapeutically effective amount” of a compound or pharmaceutical composition is that amount effective for inhibiting progression or reversing of any disease disclosed herein in a subject or a biological sample (e.g., in cells). In certain embodiments, disease progression is inhibited by about 10%, about 20%, about 30%, about 40%, about 50%, about 60%, about 70%, about 80%, about 90%, about 95%, or about 99%. In certain embodiments, the compound inhibits disease progression by at least about 25%, at least about 50%, at least about 75%, or at least about 90%. In certain embodiments of the present invention, a “therapeutically effective amount” refers to an amount of a compound or composition sufficient to reversal of disease. In certain embodiments, the disease is reversed by about 1%, about 5%, about 10%, about 20%, about 30%, about 40%, about 50%, about 60%, about 70%, about 80%, about 90%, about 95%, about 99% or any numbers and number ranges falling within these values.

As used herein, the term “lipid” refers to its conventional sense as a generic term encompassing fats, lipids, and alcohol-ether soluble constituents of protoplasm, which are insoluble in water. Lipids are composed of fats, fatty oils, essential oils, waxes, steroid, sterols, phospholipids, glycolipids, sulfolipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl and phosphatidylcholines and phosphatidylethanolamines, and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e.g., lysolecithins. Mixed formulations are also possible, particularly when the non-cationic lipids are derived from natural sources, such as lecithins (phosphatidylcholines) purified from egg yolk, bovine heart, brain, or liver, or soybean.

As used herein, the term “neutral lipids” are lipids which have a neutral net charge. “Anionic lipids” are lipid molecules which have a negative net charge. These can be selected from sterols or lipids such as cholesterol, phospholipids, lysolipids, lysophospholipids, sphingolipids or pegylated lipids with a negative net charge. “Amphiphilic lipids” are lipid molecules which exhibit both hydrophilic and hydrophobic properties. These can be selected from sterols or lipids such as cholesterol, phospholipids, lysolipids, lysophospholipids, sphingolipids or pegylated lipids with amphiphilic properties, which may have a negative, neutral, or positive net charge. Useful neutral and anionic lipids thereby include: phosphatidylserines, phosphatidylglycerols, phosphatidylinositols (not limited to a specific sugar), fatty acids, sterols containing a carboxylic acid group for example, cholesterol, phosphatidylethanolamines (PE) such as 1,2-diacyl-sn-glycero-3-phosphoethanolamines including, but not limited to 1,2-dioleoylphosphoethanolamine (DOPE), 1,2-distearoylphosphoethanolamine (DSPE), or 1,2-dihexadecoylphosphoethanolamine (DHPE), phosphatidylcholines (PC) such as 1,2-diacyl-glycero-3-phosphocholines including, but not limited to 1,2-distearoylphosphocholine (DSPC), 1,2-dipalmitoylphosphocholine (DPPC), 1,2-dimyristoylphosphocholine (DMPC), egg PC or soybean PC and sphingomyelins. The fatty acids linked to the glycerol backbone are not limited to a specific length or number of double bonds. Phospholipids may also have two different fatty acids.

As used herein, a “target” means a site to which targeted constructs bind. A target may be either in vivo or in vitro. In certain embodiments, a target may be cancer cells found in leukemias or tumors (e.g., tumors of the brain, lung (small cell and non-small cell), ovary, prostate, breast and colon as well as other carcinomas and sarcomas). In still other embodiments, a target may refer to a molecular structure to which a targeting moiety or ligand binds, such as a hapten, epitope, receptor, dsDNA fragment, carbohydrate or enzyme. A target may be a type of tissue, e.g., neuronal tissue, intestinal tissue, pancreatic tissue, liver, kidney, prostate, ovary, lung, bone marrow, or breast tissue.

As used herein, the term “targeting moiety”, as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected molecule.

As used herein, the terms “bioactive agent” and “active agent” used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

As used herein, the term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

As used herein, the terms “polypeptide,” “peptide” and “protein” generally refer to a polymer of amino acid residues. As used herein, the term also applies to amino acid polymers in which one or more amino acids are chemical analogs or modified derivatives of corresponding naturally occurring amino acids or are unnatural amino acids. The term “protein”, as generally used herein, refers to a polymer of amino acids linked to each other by peptide bonds to form a polypeptide for which the chain length is sufficient to produce tertiary and/or quaternary structure.

As used herein, the terms “nucleic acid,” “polynucleotide,” and “oligonucleotide” are used interchangeably to refer to a deoxyribonucleotide or ribonucleotide polymer, in linear or circular conformation, and in either single- or double-stranded form. These terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general and unless otherwise specified, an analog of a particular nucleotide has the same base-pairing specificity; i.e., an analog of A will base-pair with T. The term “nucleic acid” is a term of art that refers to a string of at least two base-sugar-phosphate monomeric units. Nucleotides are the monomeric units of nucleic acid polymers. The term includes deoxyribonucleic acid (DNA) and ribonucleic acid (RNA) in the form of a messenger RNA, antisense, plasmid DNA, parts of a plasmid DNA or genetic material derived from a virus. An antisense nucleic acid is a polynucleotide that interferes with the expression of a DNA and/or RNA sequence. The term nucleic acids refers to a string of at least two base-sugar-phosphate combinations. Natural nucleic acids have a phosphate backbone. Artificial nucleic acids may contain other types of backbones, but contain the same bases as natural nucleic acids. The term also includes PNAs (peptide nucleic acids), phosphorothioates, and other variants of the phosphate backbone of native nucleic acids. In an embodiment, the nucleotide sequence is provided using symbols ATCG (adenine (A), cytosine (C), guanine (G), and thymine (T)) for a DNA molecule, and provided using symbols AUCG (adenine (A), cytosine (C), guanine (G), and uracil (U)) for a RNA molecule. In an embodiment, the symbols T and U are used interchangeably in a nucleotide sequence to illustrate the DNA and RNA molecule made according to the nucleotide sequence respectively.

As used herein, “sequence identity” and “% identity,” refers to the value determined by comparing two optimally aligned sequences over a comparison window, wherein a portion of the sequence in the comparison window may comprise additions or deletions as compared to the reference sequence for optimal alignment of the two sequences. The number of positions at which identical amino acid residues occur in both sequences is determined, yielding the number of matched positions, which is divided by the total number of positions in the window of comparison and the result multiplied by 100 to yield the percentage of sequence identity. The comparison window is the entire length of the sequence being referred to unless indicated otherwise.

As used herein, “% similarity” is calculated as described for “% identity,” with the exception that the hydrophobic residues Ala, Val, Phe, Pro, Leu, Ile, Trp, Met, and Cys are similar; the basic residues Lys, Arg, and His are similar; the acidic residues Glu and Asp are similar; and the hydrophilic, uncharged residues Gln, Asn, Ser, Thr, and Tyr are similar. The remaining natural amino acid Gly is not similar to any other amino acid in this context.

As used herein, the term “protein domain” or “domain” means a region of the polypeptide of a protein. In an embodiment, a protein domain is itself folded as a compact structure independent of rest polypeptide region. In an embodiment, the same or similar protein domain within different protein can share similar or identical functions. For example, myoferlin proteins comprises multiple C2 domains and a transmembrane domain, and sometimes a Fer domain, DysF domain, or a combination thereof.

As used herein, the terms “Dys,” “DysF,” and “DysFN” are used interchangeably to refer to the DysF domain of a ferlin protein. In an embodiment, the DysFN domain comprises DysFN-1, DysFN-2, or a combination thereof, wherein DysFN-1 and DysFN-2 are the internal duplication of the DysFN domain.

Myoferlin (MYOF) is a multi-domain protein involved in receptor recycling and endocytosis. Its C2-domains such as C2A, C2B, C2C, C2D, C2E, C2F, and C2G can bind to lipid layers and change the structure of lipid packing, forming regional distorted membrane structure responsible for fusion and fission events. In addition, C2A, C2B, Fer and DysFN domains of the MYOF protein have been shown to be involved in calcium dependent trafficking and membrane repair, and the MYOF transmembrane domain is critical for protein binding to the lipid membrane and fusiogenic events. MYOF contains domains involved in receptor-mediated trafficking, which may help to reduce trafficking to the lysosome and facilitate endosomal release of payloads from nanoparticles. It has also been reported to be packaged into extracellular vesicles (EVs) and may facilitate release of EV payloads into recipient cells (1).

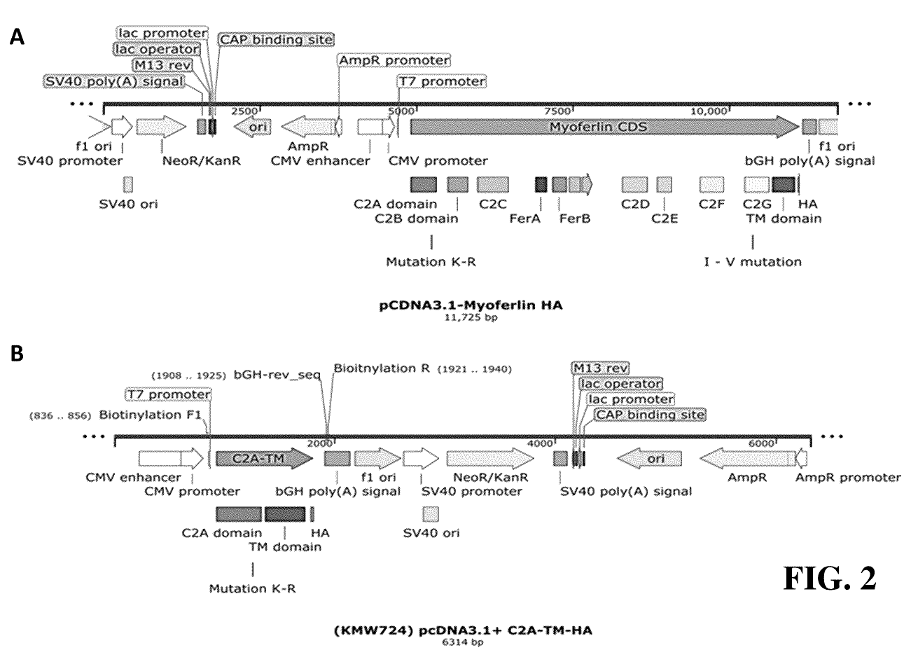

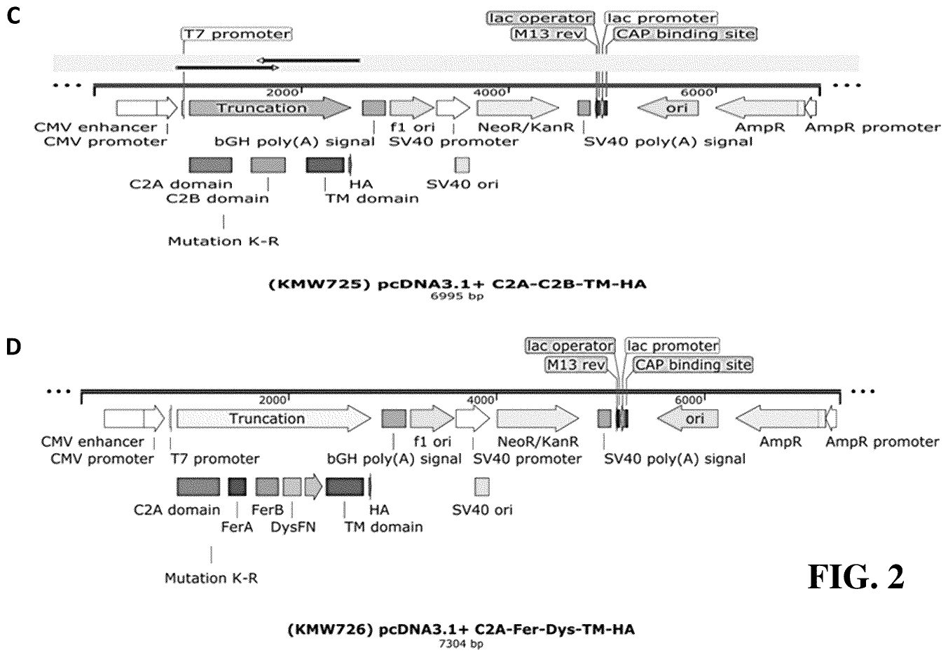

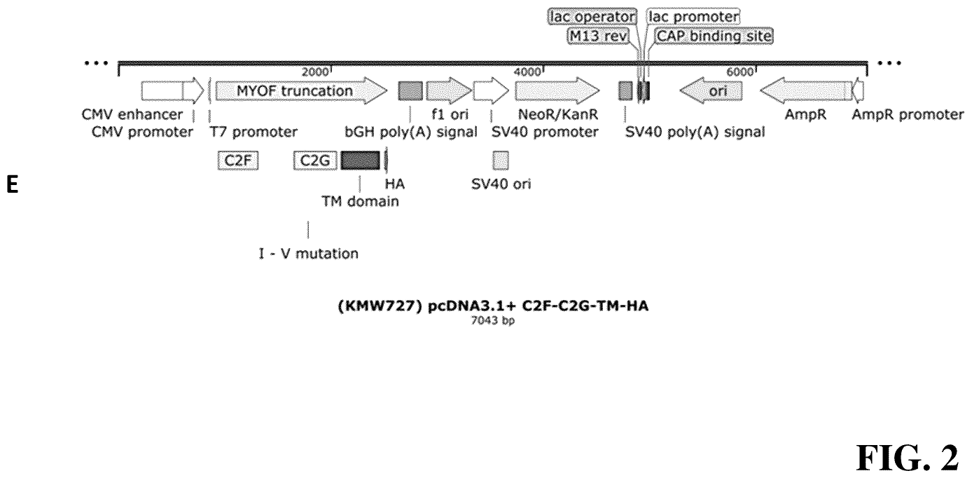

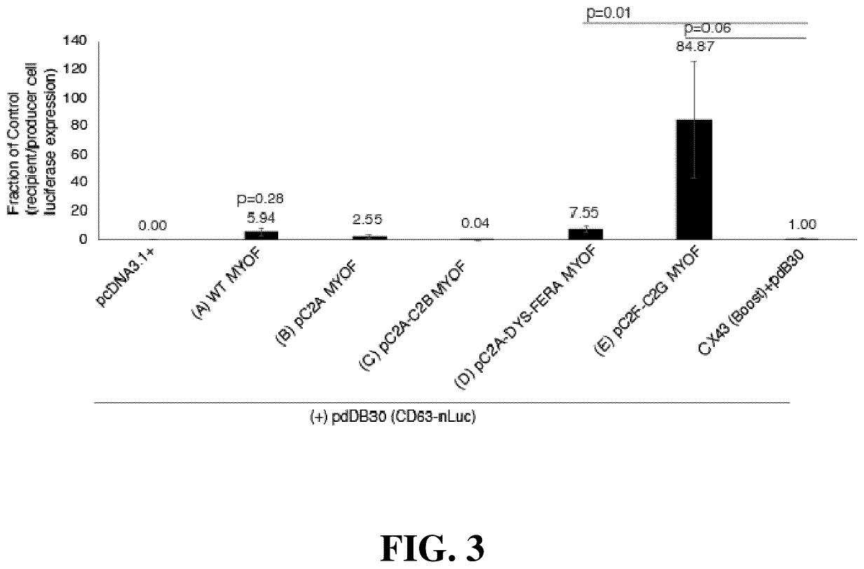

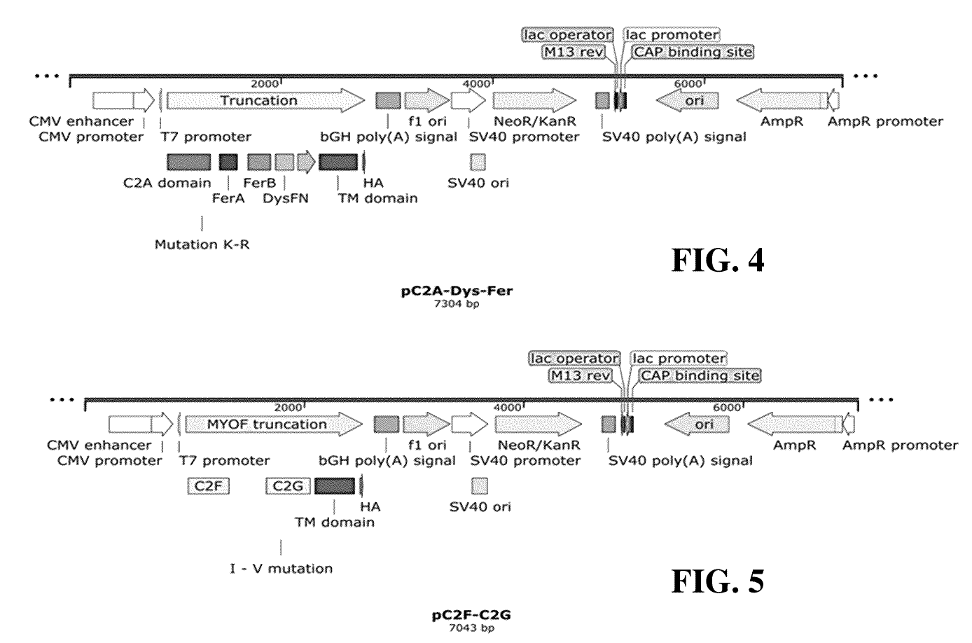

The Examples in connection with FIG. 3 show that various modified or truncated MYOF protein shown in FIG. 2B-E each facilitate extracellular vesicle (EV) mediated receptor cell uptake. Both pC2A-Dys-Fer and pC2F-C2G significantly enhanced transfer of nLuc relative to EXOtic CX43 and full-length Myoferlin as shown in FIG. 3, highlighting two unexpected results (1) that the C2F and C2G and transmembrane spanning elements are all that is required for cellular uptake of EV packaged payloads and (2) that this relatively short, ~2.5 kb sequence in pC2F-C2G MYOF can profoundly increase fusogenic uptake of nLuc. This relatively short sequence allows for this approach to be amendable to both cellular and synthetic nanosystems and could find utility in cell and gene therapies and lipid nanoparticles (LNP) to enhance both uptake and endosomal release of nucleic acid or protein payloads.

Therefore, the present invention provides a modified myoferlin protein. In an embodiment, the modified myoferlin protein of the present invention is based upon an eukaryotic myoferlin protein. In some embodiments, the modified myoferlin protein of the present invention is based upon a human myoferlin protein. In an embodiment, any embodiment of the modified myoferlin protein of the present invention is in a form comprising a recombinant protein, a polypeptide, or a polynucleotide encoding said protein. In an embodiment, the modified myoferlin protein of the present invention comprises a polypeptide comprising C2F, C2G, transmembrane (TM) domain, or a combination thereof.

In an embodiment, the modified myoferlin protein of the present invention comprises a polypeptide comprising C2F, C2G and transmembrane (TM) domains wherein the polypeptide does not comprise one or more of C2A, C2B, C2C, C2D, C2E, FerA, FerB, DysFN-1, DysFN-2 domains or a combination thereof. In an embodiment, the modified myoferlin protein of the present invention comprises a polypeptide consisting of C2F, C2G and transmembrane domains. In an embodiment, the modified myoferlin protein of the present invention comprises a polypeptide consisting of domains selected from the group consisting of C2F, C2G and transmembrane domains. In an embodiment, the amino acid sequence of the C2F domain is at least about 80%, about 85%, about 90%, about 95% or about 100% identical or similar to SEQ ID NO. 1. In an embodiment, the amino acid sequence of the C2G domain is at least about 80%, about 85%, about 90%, about 95% or about 100% identical or similar to SEQ ID NO. 2. In an embodiment, the amino acid sequence of the TM domain is at least about 80%, about 85%, about 90%, about 95% or about 100% identical or similar to SEQ ID NO. 3. In an embodiment, the C2G domain comprises an I to V mutation from the C2G domain of the wildtype human myoferlin protein to enhance the hydrophobicity of the C2G domain in the membrane and potentially offer enhanced stability. In an embodiment, any embodiment of the modified myoferlin protein of the present invention further comprises a polypeptide comprising C2A, C2B, C2C, FerA, FerB, DysFN, C2D, C2E domains, or a combination thereof.

SEQ ID NO. 1 (aa C2F):
QFRELPDSVPQECTVRIYIVRGLELQPQDNNGLCDPYIKITLGKKVIEDR
DHYIPNTLNPVFGRMYELSCYLPQEKDLKISVYDYDTFTRDEKVGETIID
LENRFLSRFGSHCGIPEEYCVSGV
SEQ ID NO. 2 (aa C2G):
PFNITPRKAKKYYLRVIIWNTKDVILDEKSITGEEMSDIYVKGWVPGNEE
NKQKTDVHYRSLDGEGNFNWRFVFPFDYLPAEQLCIVAKKEHFWSIDQTE
FRIPPRLIIQIWDNDKFSLDDYLGFLELDLRH
Bold sequence is the I to V mutation from the C2G
domain of the wildtype human myoferlin protein.
SEQ ID NO. 3 (aa TM):
PDLKAMNPLKAKTASLFEQKSMKGWWPCYAEKDGARVMAGKVEMTLEILN
EKEADERPAGKGRDEPNMNPKLDLPNRPETSFLWFTNPCKTMKFIVWRRF
KWVIIGLLFLLILLLFVAV

In an embodiment, the modified myoferlin protein of the present invention comprises a polypeptide comprising C2A, FerA, FerB, DysFN, transmembrane domain, or a combination thereof. In an embodiment, the modified myoferlin protein of the present invention comprises a polypeptide comprising C2A, FerA, FerB, DysFN and transmembrane domains wherein the polypeptide does not comprise at least one of C2B, C2C, C2D, C2E, C2F, C2G, or a combination thereof. In an embodiment, the modified myoferlin protein of the present invention comprises a polypeptide consisting of C2A, FerA, FerB, DysFN and transmembrane domains. In an embodiment, the modified myoferlin protein of the present invention comprises a polypeptide consisting of domains selected from the group consisting of C2A, FerA, FerB, DysFN and transmembrane domains. In an embodiment, the amino acid sequence of the C2A domain is at least about 80%, about 85%, about 90%, about 95% or about 100% identical or similar to SEQ ID NO. 4. In an embodiment, the amino acid sequence of the FerA domain is at least about 80%, about 85%, about 90%, about 95% or about 100% identical or similar to SEQ ID NO. 5. In an embodiment, the amino acid sequence of the FerB domain is at least about 80%, about 85%, about 90%, about 95% or about 100% identical or similar to SEQ ID NO. 6. In an embodiment, the DysFN domain comprises DysFN-1, DysFN-2, or a combination thereof. In an embodiment, the amino acid sequence of the DysFN-1 domain is at least about 80%, about 85%, about 90%, about 95% or about 100% identical or similar to SEQ ID NO. 7. In an embodiment, the amino acid sequence of the DysFN-2 domain is at least about 80%, about 85%, about 90%, about 95% or about 100% identical or similar to SEQ ID NO. 8. In an embodiment, the amino acid sequence of the transmembrane domain is at least about 80%, about 85%, about 90%, about 95% or about 100% identical or similar to SEQ ID NO. 3. In an embodiment, any embodiment of the modified myoferlin protein of the present invention further comprises a polypeptide comprising C2B, C2C, C2D, C2E, C2F, C2G domains, or a combination thereof.

SEQ ID NO. 4 (aa C2A):
MLRVIVESASNIPKTKFGKPDPIVSVIFKDEKKKTKKVDNELNPVWNEIL
EFDLRGIPLDFSSSLGIIVKDFETIGQNKLIGTATVALKDLTGDQSRSLP
YKLISLLNERGQDTGATIDLVIGYDPPSAPHPNDLS
SEQ ID NO. 5 (aa FerA):
LQTNIEALKSGIQGKIPANQLAELWLKLIDEVIEDTRYTLPLTEGKANVT
VLDTQIRK
SEQ ID NO. 6 (aa FerB):
WLDKLMQLTEEPQNSMPDIIIWMIRGEKRLAYARIPAHQVLYSTSGENAS
GKYCGKTQTIFLKYPQEKNNGP
SEQ ID NO. 7 (aa DysFN-1):
AVEKKFNSFAEGTFTVFAEMYENQALMFGKWGTSGLVGRHKFSDVTGKIK
LKREFFLP
SEQ ID NO. 8 (aa DysFN-2):
DPERSLLTEADAGHTEFTDEVYQNESRYPGGDWKPAEDTYTDANGDKAAS
PSELTCP

In an embodiment, any embodiment of the modified myoferlin protein of the present invention does not comprise a C2A domain wherein the amino acid sequence of the C2A domain is at least about 80%, about 85%, about 90%, about 95% or about 100% identical or similar to SEQ ID NO. 4. In an embodiment, any embodiment of the modified myoferlin protein of the present invention does not comprise a C2B domain wherein the amino acid sequence of the C2B domain is at least about 80%, about 85%, about 90%, about 95% or about 100% identical or similar to SEQ ID NO. 9. In an embodiment, any embodiment of the modified myoferlin protein of the present invention does not comprise a C2C domain wherein the amino acid sequence of the C2C domain is at least about 80%, about 85%, about 90%, about 95% or about 100% identical or similar to SEQ ID NO. 10. In an embodiment, any embodiment of the modified myoferlin protein of the present invention does not comprise a C2D domain wherein the amino acid sequence of the C2D domain is at least about 80%, about 85%, about 90%, about 95% or about 100% identical or similar to SEQ ID NO. 11. In an embodiment, any embodiment of the modified myoferlin protein of the present invention does not comprise a C2E domain wherein the amino acid sequence of the C2E domain is at least about 80%, about 85%, about 90%, about 95% or about 100% identical or similar to SEQ ID NO. 12. In an embodiment, any embodiment of the modified myoferlin protein of the present invention does not comprise a C2F domain wherein the amino acid sequence of the C2F domain is at least about 80%, about 85%, about 90%, about 95% or about 100% identical or similar to SEQ ID NO. 1. In an embodiment, any embodiment of the modified myoferlin protein of the present invention does not comprise a C2G domain wherein the amino acid sequence of the C2G domain is at least about 80%, about 85%, about 90%, about 95% or about 100% identical to SEQ ID NO. 2. In an embodiment, any embodiment of the modified myoferlin protein of the present invention does not comprise a FerA domain wherein the amino acid sequence of the FerA domain is at least about 80%, about 85%, about 90%, about 95% or about 100% identical or similar to SEQ ID NO. 5. In an embodiment, any embodiment of the modified myoferlin protein of the present invention does not comprise a FerB domain wherein the amino acid sequence of the FerB domain is at least about 80%, about 85%, about 90%, about 95% or about 100% identical or similar to SEQ ID NO. 6. In an embodiment, any embodiment of the modified myoferlin protein of the present invention does not comprise a DysFN-1 domain wherein the amino acid sequence of the DysFN-1 domain is at least about 80%, about 85%, about 90%, about 95% or about 100% identical or similar to SEQ ID NO. 7. In an embodiment, any embodiment of the modified myoferlin protein of the present invention does not comprise a DysFN-2 domain wherein the amino acid sequence of the DysFN-2 domain is at least about 80%, about 85%, about 90%, about 95% or about 100% identical or similar to SEQ ID NO. 8. In an embodiment, any embodiment of the modified myoferlin protein of the present invention does not comprise a transmembrane domain wherein the amino acid sequence of the transmembrane domain is at least about 80%, about 85%, about 90%, about 95% or about 100% identical or similar to SEQ ID NO 3

SEQ ID NO. 9 (aa C2B):
PQDFQIRVRVIEGRQLSGNNIRPVVKVHVCGQTHRTRIKRGNNPFFDELF
FYNVNMTPSELMDEIISIRVYNSHSLRADCLMGEFKIDVGFVYDEPGHAV
MRKWLLLNDP
SEQ ID NO. 10 (aa C2C):
TFLLKIYRAEDIPQMDDAFSQTVKEIFGGNADKKNLVDPFVEVSFAGKKV
CTNIIEKNANPEWNQVVNLQIKFPSVCEKIKLTIYDWDRLTKNDVVGTTY
LHLSKIAASGGEVEDFSSSGTGAASYTVNTGETEVGFVPTFGPCYLNLYG
SPREYTGFPDPYDE
SEQ ID NO. 11 (aa C2D):
TPIVSCNFDRVYIYHLRCYVYQARNLLALDKDSFSDPYAHICFLHRSKTT
EIIHSTLNPTWDQTIIFDEVEIYGEPQTVLQNPPKVIMELFDNDQVGKDE
FLGRSIFSPVVKLNSEMDITPKLLWHPVMNGDKA
SEQ ID NO. 12 (aa C2E):
RNMKNFQMASITSPSLVVECGGERVESVVIKNLKKTPNFPSSVLFMKVFL
PKEELYMPPLVIKVIDHRQFGRKPVVG

The present invention also provides a polynucleotide that encodes a modified myoferlin protein comprising C2F, C2G, transmembrane domain, or a combination thereof. In an embodiment, the polynucleotide of the present invention encodes a modified myoferlin protein comprising C2F, C2G and transmembrane domains wherein the modified myoferlin protein does not comprise at least one of C2A, C2B, C2C, C2D, C2E, FerA, FerB, DysFN-1, DysFN-2 domains or a combination thereof. In an embodiment, the polynucleotide of the present invention encodes a modified myoferlin protein consisting of C2F, C2G and transmembrane domains. In an embodiment, the polynucleotide of the present invention encodes a modified myoferlin protein consisting of one or more domains selected from the group consisting of C2F, C2G and transmembrane domains. In an embodiment, the nucleotide sequence encoding the C2F domain is at least about 80%, about 85%, about 90%, about 95% or about 100% identical to SEQ ID NO. 13. In an embodiment, the nucleotide sequence encoding the C2G domain is at least about 80%, about 85%, about 90%, about 95% or about 100% identical to SEQ ID NO. 14. In an embodiment, the nucleotide sequence encoding the transmembrane domain is at least about 80%, about 85%, about 90%, about 95% or about 100% identical to SEQ ID NO. 15. In an embodiment, the C2G domain comprises an I-V mutation from the C2G domain of the wildtype human myoferlin protein to enhance the hydrophobicity of the C2G domain in the membrane and potentially offer enhanced stability. In an embodiment, any embodiment of the polynucleotide of the present invention encoding a modified myoferlin protein further encodes C2A, C2B, C2C, FerA, FerB, DysFN, C2D, C2E domains, or a combination thereof.

SEQ ID NO. 13 (ntC2F):
CAGTTTCGGGAATTACCTGACAGCGTCCCACAGGAATGCACGGTTAGGAT
TTACATTGTTCGAGGCTTAGAGCTCCAGCCCCAGGACAACAATGGCCTGT
GTGACCCTTACATAAAAATAACACTGGGCAAAAAAGTCATTGAAGACCGA
GATCACTACATTCCCAACACTCTCAACCCAGTCTTTGGCAGGATGTACGA
ACTGAGCTGCTACTTACCTCAAGAAAAAGACCTGAAAATTTCTGTCTATG
ATTATGACACCTTTACCCGGGATGAAAAAGTAGGAGAGACAATTATTGAT
CTGGAAAACCGATTCCTTTCCCGCTTTGGGTCCCACTGCGGCATACCAGA
GGAGTACTGTGTTTCTGGAGTC
SEQ ID NO. 14 (nt C2G):
CCTTTCAACATCACACCCCGGAAAGCCAAGAAATACTACCTGCGTGTGAT
CATCTGGAACACCAAGGATGTTATCTTGGATGAGAAAAGCATCACAGGAG
AGGAAATGAGTGACATCTACGTCAAAGGCTGGGTTCCTGGCAATGAAGAA
AACAAACAGAAAACAGATGTCCATTACAGATCTTTGGATGGTGAAGGGAA
TTTTAACTGGCGATTTGTTTTCCCGTTTGACTACCTTCCAGCCGAACAAC
TCTGTATCGTTGCGAAAAAAGAGCATTTCTGGAGTATTGACCAAACGGAA
TTTCGAATCCCACCCAGGCTGATCATTCAGATATGGGACAATGACAAGTT
TTCTCTGGATGACTACTTGGGTTTCCTAGAACTTGACTTGCGTCAC

Bold sequence is the I to V mutation from the C2G domain of the wildtype human myoferlin protein.

SEQ ID NO. 15 (nt TM):
CCGGACCTCAAAGCCATGAACCCCCTTAAAGCCAAGACAGCCTCCCTCTT
TGAGCAGAAGTCCATGAAAGGATGGTGGCCATGCTACGCAGAGAAAGATG
GCGCCCGCGTAATGGCTGGGAAAGTGGAGATGACATTGGAAATCCTCAAC
GAGAAGGAGGCCGACGAGAGGCCAGCCGGGAAGGGGCGGGACGAACCCAA
CATGAACCCCAAGCTGGACTTACCAAATCGACCAGAAACCTCCTTCCTCT
GGTTCACCAACCCATGCAAGACCATGAAGTTCATCGTGTGGCGCCGCTTT
AAGTGGGTCATCATCGGCTTGCTGTTCCTGCTTATCCTGCTGCTCTTCGT
GGCCGTG

In an embodiment, the polynucleotide of the present invention encodes a myoferlin protein comprising C2A, FerA, FerB, DysFN, transmembrane domain, or a combination thereof. In an embodiment, the polynucleotide of the present invention encodes a modified myoferlin protein comprising C2A, FerA, FerB, DysFN and transmembrane domains wherein the modified myoferlin protein does not comprise at least one of C2B, C2C, C2D, C2E, C2F, C2G domains or a combination thereof. In an embodiment, the polynucleotide of the present invention encodes a modified myoferlin protein consisting of C2A, FerA, FerB, DysFN and transmembrane domains. In an embodiment, the polynucleotide of the present invention encodes a modified myoferlin protein consisting of one or more domains selected from the group consisting of C2A, FerA, FerB, DysFN and transmembrane domains. In an embodiment, the nucleotide sequence encoding the C2A domain is at least about 80%, about 85%, about 90%, about 95% or about 100% identical to SEQ ID NO. 16. In an embodiment, the nucleotide sequence encoding the FerA domain is at least about 80%, about 85%, about 90%, about 95% or about 100% identical to SEQ ID NO. 17. In an embodiment, the nucleotide sequence encoding the FerB domain is at least about 80%, about 85%, about 90%, about 95% or about 100% identical to SEQ ID NO. 18. In an embodiment, the DysFN domain comprises DysFN-1, DysFN-2, or a combination thereof. In an embodiment, the nucleotide sequence encoding the DysFN-1 domain is at least about 80%, about 85%, about 90%, about 95% or about 100% identical to SEQ ID NO. 19. In an embodiment, the nucleotide sequence encoding the DysFN-2 domain is at least about 80%, about 85%, about 90%, about 95% or about 100% identical to SEQ ID NO. 20. In an embodiment, the nucleotide sequence encoding the transmembrane domain is at least about 80%, about 85%, about 90%, about 95% or about 100% identical to SEQ ID NO. 15. In an embodiment, any embodiment of the polynucleotide of the present invention encoding a modified myoferlin protein further encodes C2B, C2C, C2D, C2E, C2F, C2G domains, or a combination thereof of the modified myoferlin protein.

SEQ ID NO. 16 (nt C2A):
ATGCTGCGAGTGATTGTGGAATCTGCCAGCAATATCCCTAAAACGAAATT
TGGCAAGCCGGATCCTATTGTTTCTGTCATTTTTAAGGATGAGAAAAAGA
AAACAAAGAAAGTTGATAATGAATTGAACCCTGTCTGGAATGAGATTTTG
GAGTTTGACTTGAGGGGTATACCACTGGACTTTTCATCTTCCCTTGGGAT
TATTGTGAAAGATTTTGAGACAATTGGACAAAATAAATTAATTGGCACGG
CGACTGTAGCCCTGAAGGACCTGACTGGTGACCAGAGCAGATCCCTGCCG
TACAAGCTGATCTCCCTGCTAAATGAAAGAGGGCAAGATACTGGGGCCAC
CATTGACTTGGTGATCGGCTATGATCCGCCTTCTGCTCCACATCCAAATG
ACCTGAGC
SEQ ID NO. 17 (nt FerA):
CTGCAAACAAATATAGAAGCTCTAAAATCAGGGATACAAGGTAAAATTCC
TGCAAACCAGCTGGCTGAATTGTGGCTGAAGCTGATAGATGAAGTTATAG
AAGACACGAGATACACGTTGCCTCTCACAGAAGGAAAAGCCAACGTCACA
GTTCTCGATACTCAGATCCGAAAG
SEQ ID NO. 18 (nt FerB):
TGGCTTGATAAATTAATGCAGCTGACTGAAGAGCCACAGAACAGCATGCC
TGACATCATCATCTGGATGATCCGGGGAGAGAAGAGACTGGCCTATGCAC
GAATTCCCGCACATCAGGTCTTGTACTCCACCAGTGGTGAGAATGCATCT
GGAAAATACTGTGGGAAAACCCAAACCATCTTTCTGAAGTATCCACAGGA
GAAAAACAACGGGCCA
SEQ ID NO. 19 (nt DysFN-1):
GCTGTGGAGAAGAAGTTTAACAGCTTCGCAGAAGGAACTTTCACCGTCTT
TGCTGAAATGTATGAAAATCAAGCTCTCATGTTTGGAAAATGGGGTACTT
CTGGATTAGTAGGACGTCATAAGTTTTCTGATGTCACAGGAAAAATAAAA
CTCAAGAGGGAATTTTTTCTGCCT
SEQ ID NO. 20 (nt DysFN-2):
GATCCTGAAAGAAGCTTGCTGACTGAGGCAGATGCAGGTCACACGGAGTT
CACTGATGAAGTCTACCAGAACGAGAGCCGCTACCCCGGGGGCGACTGGA
AGCCGGCCGAGGACACCTACACGGATGCGAACGGCGATAAAGCAGCATCA
CCCAGCGAGTTGACTTGTCCT

In an embodiment, any embodiment of the polynucleotide encoding a myoferlin protein of the present invention does not encode the C2A domain wherein the nucleotide sequence of the C2A domain is at least about 80%, about 85%, about 90%, about 95% or about 100% identical to SEQ ID NO. 16. In an embodiment, any embodiment of the polynucleotide encoding a myoferlin protein of the present invention does not encode the C2B domain wherein the nucleotide sequence of the C2B domain is at least about 80%, about 85%, about 90%, about 95% or about 100% identical to SEQ ID NO. 21. In an embodiment, any embodiment of the polynucleotide encoding a myoferlin protein of the present invention does not encode the C2C domain wherein the nucleotide sequence of the C2C domain is at least about 80%, about 85%, about 90%, about 95% or about 100% identical to SEQ ID NO. 22. In an embodiment, any embodiment of the polynucleotide encoding a myoferlin protein of the present invention does not encode the C2D domain wherein the nucleotide sequence of the C2D domain is at least about 80%, about 85%, about 90%, about 95% or about 100% identical to SEQ ID NO. 23. In an embodiment, any embodiment of the polynucleotide encoding a myoferlin protein of the present invention does not encode the C2E domain wherein the nucleotide sequence of the C2E domain is at least about 80%, about 85%, about 90%, about 95% or about 100% identical to SEQ ID NO. 24. In an embodiment, any embodiment of the polynucleotide encoding a myoferlin protein of the present invention does not encode the C2F domain wherein the nucleotide sequence of the C2F domain is at least about 80%, about 85%, about 90%, about 95% or about 100% identical to SEQ ID NO. 13. In an embodiment, any embodiment of the polynucleotide encoding a myoferlin protein of the present invention does not encode the C2G domain wherein the nucleotide sequence of the C2G domain is at least about 80%, about 85%, about 90%, about 95% or about 100% identical to SEQ ID NO. 14. In an embodiment, any embodiment of the polynucleotide encoding a myoferlin protein of the present invention does not encode the FerA domain wherein the nucleotide sequence of the FerA domain is at least about 80%, about 85%, about 90%, about 95% or about 100% identical to SEQ ID NO. 17. In an embodiment, any embodiment of the polynucleotide encoding a myoferlin protein of the present invention does not encode the FerB domain wherein the nucleotide sequence of the FerB domain is at least about 80%, about 85%, about 90%, about 95% or about 100% identical to SEQ ID NO. 18. In an embodiment, any embodiment of the polynucleotide encoding a myoferlin protein of the present invention does not encode the DysFN-1 domain wherein the nucleotide sequence of the DysFN-1 domain is at least about 80%, about 85%, about 90%, about 95% or about 100% identical to SEQ ID NO. 19. In an embodiment, any embodiment of the polynucleotide encoding a myoferlin protein of the present invention does not comprise the DysFN-2 domain wherein the nucleotide sequence of the DysFN-2 domain is at least about 80%, about 85%, about 90%, about 95% or about 100% identical to SEQ ID NO. 20. In an embodiment, any embodiment of the polynucleotide encoding a myoferlin protein of the present invention does not encode the transmembrane domain wherein the nucleotide sequence of the transmembrane domain is at least about 80%, about 85%, about 90%, about 95% or about 100% identical to SEQ ID NO. 15.

SEQ ID NO. 21 (nt C2B):
CCACAGGACTTCCAGATCCGCGTCCGAGTGATTGAGGGCCGACAGTTAAG
TGGCAACAACATAAGGCCTGTGGTCAAAGTTCACGTCTGTGGCCAGACAC
ACCGAACAAGAATCAAGAGAGGAAACAACCCTTTTTTTGATGAGTTGTTT
TTCTACAATGTCAACATGACCCCTTCTGAATTGATGGATGAGATCATCAG
CATCCGGGTTTATAATTCTCACTCTCTGCGGGCAGATTGTCTGATGGGGG
AATTTAAGATTGATGTTGGATTTGTTTATGATGAACCTGGCCATGCTGTC
ATGAGAAAGTGGCTTCTTCTCAATGACCCG
SEQ ID NO. 22 (nt C2C):
ACCTTCTTGCTGAAAATCTACCGAGCTGAGGACATCCCCCAGATGGATGA
TGCCTTCTCACAGACAGTAAAGGAAATATTTGGAGGCAATGCAGATAAGA
AAAATCTCGTGGATCCTTTTGTAGAAGTTTCCTTTGCTGGAAAAAAGGTT
TGTACAAACATAATTGAGAAGAATGCAAACCCAGAGTGGAATCAGGTCGT
CAATCTTCAGATCAAGTTTCCTTCAGTGTGTGAAAAAATAAAACTAACAA
TATATGACTGGGACCGTCTTACTAAAAATGATGTAGTTGGAACAACATAT
CTACACCTCTCTAAAATTGCTGCCTCTGGTGGGGAAGTGGAAGATTTCTC
ATCTTCGGGAACTGGGGCTGCATCATATACAGTAAACACAGGAGAAACAG
AGGTAGGCTTTGTTCCAACGTTTGGACCTTGTTACCTGAATCTTTATGGA
AGCCCCAGAGAGTACACGGGATTCCCAGACCCCTATGATGAG
SEQ ID NO. 23 (nt C2D):
ACCCCCATTGTTTCCTGCAATTTTGACAGAGTCTACATCTACCATCTGCG
CTGCTATGTCTATCAAGCCAGAAACCTCTTGGCTTTAGATAAGGATAGCT
TTTCAGATCCATATGCTCATATCTGTTTCCTCCATCGGAGCAAAACCACT
GAGATCATCCATTCAACCCTGAATCCCACGTGGGACCAAACAATTATATT
CGATGAAGTTGAAATCTATGGGGAACCCCAAACAGTTCTACAGAATCCAC
CCAAAGTTATCATGGAACTTTTTGACAATGACCAAGTGGGCAAAGATGAA
TTTTTAGGACGAAGCATTTTCTCTCCTGTGGTGAAACTGAACTCAGAAAT
GGACATCACACCCAAACTTCTCTGGCACCCAGTAATGAATGGAGACAAAG
CC
SEQ ID NO. 24 (nt C2E):
AGAAATATGAAAAACTTCCAGATGGCTTCTATCACATCCCCCAGTCTTGT
TGTGGAGTGTGGAGGAGAAAGGGTGGAATCGGTGGTGATCAAAAACCTTA
AGAAGACACCCAACTTTCCAAGTTCTGTTCTCTTCATGAAAGTGTTCTTG
CCCAAGGAGGAATTGTACATGCCCCCACTGGTGATCAAGGTCATCGACCA
CAGGCAGTTTGGGCGGAAGCCTGTCGTCGGC

In an embodiment, the modified myoferlin protein of the present invention comprises a truncated wild type human myoferlin protein. In an embodiment, the modified myoferlin protein of the present invention comprises at least a C2 domain, wherein the C2 domain comprises C2A, C2B, C2C, C2D, C2E, C2F, or C2G. In an embodiment, the modified myoferlin protein of the present invention comprises at least two C2 domains, wherein the C2 domain comprises C2A, C2B, C2C, C2D, C2E, C2F, or C2G. In an embodiment, the modified myoferlin protein of the present invention comprises a C2 domain, a FerA domain, a FerB domain, a DysF domain, a transmembrane domain, or a combination thereof. In an embodiment, the DysFN domain comprises DysFN-1, DysFN-2, or a combination thereof. In an embodiment, the modified myoferlin protein of the present invention comprises a myoferlin C2A domain, a myoferlin C2F domain, a myoferlin C2G domain, a myoferlin DysF domain, a myoferlin FerA domain, a myoferlin FerB domain, a myoferlin transmembrane domain, or a combination thereof.

The present invention further provides a dual protein comprising any embodiment of the modified myoferlin protein of the present invention fused to a connexin 43 protein. In an embodiment, the connexin 43 protein comprises a S368A mutation. An embodiment of the dual protein is shown in FIG. 6. The dual protein of the present invention can be used to substantially enhance the efficacy and delivery of RNA and nucleic acid payloads. In an embodiment, the dual protein of the present invention comprises a first polypeptide comprising an embodiment of the modified myoferlin protein of the present invention and a second polypeptide comprising a connexin 43 protein. In an embodiment, the first polypeptide comprises an amino acid sequence at least about 80%, about 85%, about 90%, about 95% or about 100% identical or similar to SEQ ID NO. 25. In an embodiment, the second polypeptide comprises an amino acid sequence at least about 80%, about 85%, about 90%, about 95% or about 100% identical or similar to SEQ ID NO. 26. In an embodiment, the dual protein of the present invention further comprises a third polypeptide linking the first polypeptide comprising the modified myoferlin protein of the present invention and the second polypeptide comprising the connexin 43 protein. In an embodiment, the third polypeptide linking the first polypeptide and the second polypeptide allows the first polypeptide and the second polypeptide to each form a tertiary structure similar or identical to the tertiary structure formed by each polypeptide in its separated form.

SEQ ID NO. 25 (aa C2F-C2G):
MVPAPPRQFRELPDSVPQECTVRIYIVRGLELQPQDNNGLCDPYIKITLG
KKVIEDRDHYIPNTLNPVFGRMYELSCYLPQEKDLKISVYDYDTFTRDEK
VGETIIDLENRFLSRFGSHCGIPEEYCVSGVNTWRDQLRPTQLLQNVARF
KGFPQPILSEDGSRIRYGGRDYSLDEFEANKILHQHLGAPEERLALHILR
TQGLVPEHVETRTLHSTFQPNISQGKLQMWVDVFPKSLGPPGPPFNITPR
KAKKYYLRVIIWNTKDVILDEKSITGEEMSDIYVKGWVPGNEENKQKTDV
HYRSLDGEGNFNWRFVFPFDYLPAEQLCIVAKKEHFWSIDQTEFRIPPRL
IIQIWDNDKFSLDDYLGFLELDLRHTIIPAKSPEKCRLDMIPDLKAMNPL
KAKTASLFEQKSMKGWWPCYAEKDGARVMAGKVEMTLEILNEKEADERPA
GKGRDEPNMNPKLDLPNRPETSFLWFTNPCKTMKFIVWRRFKWVIIGLLF
LLILLLFVAVLLYSLPNYLSMKIVKPNVYPYDVPDYA
SEQ ID NO. 26 (aa connexin 43):
MATTMGDWSALGKLLDKVQAYSTAGGKVWLSVLFIFRILLLGTAVESAWG
DEQSAFRCNTQQPGCENVCYDKSFPISHVRFWVLQIIFVSVPTLLYLAHV
FYVMRKEEKLNKKEEELKVAQTDGVNVDMHLKQIEIKKFKYGIEEHGKVK
MRGGLLRTYIISILFKSIFEVAFLLIQWYIYGFSLSAVYTCKRDPCPHQV
DCFLSRPTEKTIFIIFMLVVSLVSLALNIIELFYVFFKGVKDRVKGKSDP
YHATSGALSPAKDCGSQKYAYFNGCSSPTAPLSPMSPPGYKLVTGDRNNS
SCRNYNKQASEQNWANYSAEQNRMGQAGSTISNSHAQPFDFPDDNQNSKK
LAAGHELQPLAIVDQRPSSRAASRASSRPRPDDLEI

The present invention also provides a dual protein expressing endosomal release plasmid comprising a plasmid encoding any embodiment of the modified myoferlin protein of the present invention and connexin 43. In an embodiment, the connexin 43 protein comprises a S368A mutation. An embodiment is shown in FIG. 6. The dual protein of the present invention can be used to greatly enhance the efficacy and delivery of RNA and nucleic acid payloads. In an embodiment, the dual protein expressing endosomal release plasmid comprises a first polynucleotide comprising an embodiment of the modified myoferlin protein of the present invention and a second polynucleotide comprising a connexin 43 protein. In an embodiment, the dual protein expressing endosomal release plasmid further comprises a third polynucleotide linking the first polynucleotide encoding the modified myoferlin protein and the second polynucleotide encoding the connexin 43 protein. In an embodiment, the nucleotide sequence of the dual protein of the present invention is at least about 80%, about 85%, about 90%, about 95% or about 100% identical SEQ ID NO. 27.

SEQ ID NO. 27 (nt myo-cnx43):
ATGGTGCCAGCCCCTCCCAGACAGTTTCGGGAATTACCTGACAGCGTCCCACAGG
AATGCACGGTTAGGATTTACATTGTTCGAGGCTTAGAGCTCCAGCCCCAGGACAA
CAATGGCCTGTGTGACCCTTACATAAAAATAACACTGGGCAAAAAAGTCATTGA
AGACCGAGATCACTACATTCCCAACACTCTCAACCCAGTCTTTGGCAGGATGTAC
GAACTGAGCTGCTACTTACCTCAAGAAAAAGACCTGAAAATTTCTGTCTATGATT
ATGACACCTTTACCCGGGATGAAAAAGTAGGAGAGACAATTATTGATCTGGAAA
ACCGATTCCTTTCCCGCTTTGGGTCCCACTGCGGCATACCAGAGGAGTACTGTGT
TTCTGGAGTCAATACCTGGCGAGATCAACTGAGACCAACACAGCTGCTTCAAAAT
GTCGCCAGATTCAAAGGCTTCCCACAACCCATCCTTTCCGAAGATGGGAGTAGAA
TCAGATATGGAGGACGAGACTACAGCTTGGATGAATTTGAAGCCAACAAAATCC
TGCACCAGCACCTCGGGGCCCCTGAAGAGCGGCTTGCTCTTCACATCCTCAGGAC
TCAGGGGCTGGTCCCTGAGCACGTGGAAACAAGGACTTTGCACAGCACCTTCCA
GCCCAACATTTCCCAGGGAAAACTTCAGATGTGGGTGGATGTTTTCCCCAAGAGT
TTGGGGCCACCAGGCCCTCCTTTCAACATCACACCCCGGAAAGCCAAGAAATAC
TACCTGCGTGTGATCATCTGGAACACCAAGGATGTTATCTTGGATGAGAAAAGCA
TCACAGGAGAGGAAATGAGTGACATCTACGTCAAAGGCTGGGTTCCTGGCAATG
AAGAAAACAAACAGAAAACAGATGTCCATTACAGATCTTTGGATGGTGAAGGGA
ATTTTAACTGGCGATTTGTTTTCCCGTTTGACTACCTTCCAGCCGAACAACTCTGT
ATCGTTGCGAAAAAAGAGCATTTCTGGAGTATTGACCAAACGGAATTTCGAATCC
CACCCAGGCTGATCATTCAGATATGGGACAATGACAAGTTTTCTCTGGATGACTA
CTTGGGTTTCCTAGAACTTGACTTGCGTCACACGATCATTCCTGCAAAATCACCA
GAGAAATGCAGGTTGGACATGATTCCGGACCTCAAAGCCATGAACCCCCTTAAA
GCCAAGACAGCCTCCCTCTTTGAGCAGAAGTCCATGAAAGGATGGTGGCCATGC
TACGCAGAGAAAGATGGCGCCCGCGTAATGGCTGGGAAAGTGGAGATGACATTG
GAAATCCTCAACGAGAAGGAGGCCGACGAGAGGCCAGCCGGGAAGGGGCGGGA
CGAACCCAACATGAACCCCAAGCTGGACTTACCAAATCGACCAGAAACCTCCTT
CCTCTGGTTCACCAACCCATGCAAGACCATGAAGTTCATCGTGTGGCGCCGCTTT
AAGTGGGTCATCATCGGCTTGCTGTTCCTGCTTATCCTGCTGCTCTTCGTGGCCGT
GCTCCTCTACTCTTTGCCGAACTATTTGTCAATGAAGATTGTAAAGCCAAATGTG
TACCCATACGACGTCCCAGACTACGCTTAGGCCCCTCTCCCTCCCCCCCCCCTA
ACGTTACTGGCCGAAGCCGCTTGGAATAAGGCCGGTGTGCGTTTGTCTATA
TGTTATTTTCCACCATATTGCCGTCTTTTGGCAATGTGAGGGCCCGGAAACC
TGGCCCTGTCTTCTTGACGAGCATTCCTAGGGGTCTTTCCCCTCTCGCCAAA
GGAATGCAAGGTCTGTTGAATGTCGTGAAGGAAGCAGTTCCTCTGGAAGCT
TCTTGAAGACAAACAACGTCTGTAGCGACCCTTTGCAGGCAGCGGAACCCCCC
ACCTGGCGACAGGTGCCTCTGCGGCCAAAAGCCACGTGTATAAGATACACCTGC
AAAGGCGGCACAACCCCAGTGCCACGTTGTGAGTTGGATAGTTGTGGAAAGAGT
CAAATGGCTCTCCTCAAGCGTATTCAACAAGGGGCTGAAGGATGCCCAGAAGGT
ACCCCATTGTATGGGATCTGATCTGGGGCCTCGGTGCACATGCTTTACATGTGTTT
AGTCGAGGTTAAAAAAACGTCTAGGCCCCCCGAACCACGGGGACGTGGTTTTCC
TTTGAAAAACACGATGATAATATGGCCACAACCATGGGTGACTGGAGCGCCTTA
GGCAAACTCCTTGACAAGGTTCAAGCCTACTCAACTGCTGGAGGGAAGGTGTGG
CTGTCAGTACTTTTCATTTTCCGAATCCTGCTGCTGGGGACAGCGGTTGAGTCAG
CCTGGGGAGATGAGCAGTCTGCCTTTCGTTGTAACACTCAGCAACCTGGTTGTGA
AAATGTCTGCTATGACAAGTCTTTCCCAATCTCTCATGTGCGCTTCTGGGTCCTGC
AGATCATATTTGTGTCTGTACCCACACTCTTGTACCTGGCTCATGTGTTCTATGTG
ATGCGAAAGGAAGAGAAACTGAACAAGAAAGAGGAAGAACTCAAGGTTGCCCA
AACTGATGGTGTCAATGTGGACATGCACTTGAAGCAGATTGAGATAAAGAAGTT
CAAGTACGGTATTGAAGAGCATGGTAAGGTGAAAATGCGAGGGGGGTTGCTGCG
AACCTACATCATCAGTATCCTCTTCAAGTCTATCTTTGAGGTGGCCTTCTTGCTGA
TCCAGTGGTACATCTATGGATTCAGCTTGAGTGCTGTTTACACTTGCAAAAGAGA
TCCCTGCCCACATCAGGTGGACTGTTTCCTCTCTCGCCCCACGGAGAAAACCATC
TTCATCATCTTCATGCTGGTGGTGTCCTTGGTGTCCCTGGCCTTGAATATCATTGA
ACTCTTCTATGTTTTCTTCAAGGGCGTTAAGGATCGGGTTAAGGGAAAGAGCGAC
CCTTACCATGCGACCAGTGGTGCGCTGAGCCCTGCCAAAGACTGTGGGTCTCAAA
AATATGCTTATTTCAATGGCTGCTCCTCACCAACCGCTCCCCTCTCGCCTATGTCT
CCTCCTGGGTACAAGCTGGTTACTGGCGACAGAAACAATTCTTCTTGCCGCAATT
ACAACAAGCAAGCAAGTGAGCAAAACTGGGCTAATTACAGTGCAGAACAAAATC
GAATGGGGCAGGCGGGAAGCACCATCTCTAACTCCCATGCACAGCCTTTTGATTT
CCCCGATGATAACCAGAATTCAAAAAAACTAGCTGCTGGACATGAATTACAGCC
ACTAGCCATTGTGGACCAGCGACCTTCAAGCAGAGCCGCCAGTCGTGCCAGCAG
CAGACCTCGGCCTGATGACCTGGAGATCTGA

Bold sequence indicates the internal ribosome entry site (IRES) between the modified myoferlin protein and the connexin 43 protein.

The present invention further provides a modified myoferlin-encapsulating nanoparticle comprising a nanoparticle and any embodiment of the modified myoferlin protein of the present invention or the dual protein of the present invention wherein the nanoparticle encapsulates the any embodiment of the modified myoferlin protein or dual protein of the present invention. In an embodiment, the modified myoferlin-encapsulating nanoparticle of the present invention further encapsulates a payload. In an embodiment, the nanoparticle comprises liposome, lipid nanoparticle, polymer nanoparticle, lipid-polymer hybrid nanoparticle, virus-like particle or exosome. In an embodiment, the nanoparticle comprises one or more conjugates for targeted delivery to a recipient cell in a subject. The selection of the one or more conjugate can be dependent on the cell type or the organ of a subject in need thereof for the delivery of the nanoparticles of the present invention.

In an embodiment, any embodiment of the modified myoferlin-encapsulating nanoparticle of the present invention preferably comprises neutral lipids such as but not limited to L-α-phosphatidylcholine, hydrogenated soybean phosphatidylcholine (HSPC), 1,2-dioeoyl-sn-glycero-3-phosphocholine (DOPC), 1,2-dioleoylphosphoethanolamine (DOPE), 1,2-distearoylphosphoethanolamine (DSPE), and/or polyethyleneglycol-derivated distearoylphosphatidylethanolamine (PEG-DSPE), or 1,2-dihexadecoylphosphoethanolamine (DHPE), phosphatidylcholine (PC) such as 1,2-diacyl-glycero-3-phosphocholines including but not limited to 1,2-distearoylphosphocholine (DSPC), 1,2-dipalmitoylphosphocholine (DPPC), and 1,2-dimyristoyl-sn-glycero-3-phosphocholine (DMPC).

In an embodiment, any embodiment of the modified myoferlin-encapsulating nanoparticle of the present invention further comprises charged lipid comprising anionic lipids which may comprise phosphatidylglycerol, cardioplipin, diacylphosphatidylserine, diacylphosphatidic acid, lysylphosphatidylglycerol, egg L-α-phosphatidylglycerol (EPG), 1,2-dimyristoyl-sn-glycero-3-phosphoglycerol (DMPG), 1,2-dipalmitoyl-sn-glycero-3-phosphoglycerol (DPPG), 1,2-dioleoyl-sn-glycero-3-phospho-(1′-rac-glycerol) (DOPG), 1,2-dioeoyl-sn-glycero-3-phosphate (DOPA), 1,2-dioleoyl-sn-glycero-3-phospho-L-serine (DOPS), and other anionic modifying groups joined to neutral lipids. The charge on each anionic lipid may be in the range of about −1 to −5 eV, about −1 to −3 eV, about −1 to −2 eV. In an embodiment, the payload comprises a therapeutic active agent, a prophylactic agent, a diagnostic agent, a nutritional agent, or a combination thereof. In an embodiment, the payload comprises a polynucleotide, a polypeptide, a recombinant protein, a small-molecule drug, or a combination thereof. In an embodiment, the polynucleotide comprises a siRNA, an asRNA, a circular RNA, a DNA, a mRNA, a lncRNA, a shRNA, a microRNA, a guide RNA (gRNA) an antisense oligonucleotide, a ribozyme, a plasmid and/or an immune stimulating nucleic acid, etc. . . . . In an embodiment, the therapeutic agent comprises chemotherapeutic agents including, but are not limited to alkylating agents, antimetabolites, anthracyclines, plant alkaloids, topoisomerase inhibitors, or other antitumor agents. In an embodiment, the therapeutic agent comprises chemotherapeutic agents including, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab, cetuximab, and rituximab, bevacizumab, and combinations thereof.

In an embodiment, less than about 0.1%, about 0.2%, about 0.3%, about 0.4%, about 0.5%, about 0.6%, about 0.7%, about 0.8%, about 0.9%, about 1%, about 5%, about 10%, about 20%, about 30%, about 40%, or about 50% of the payload is released from the nanoparticle prior to the delivery or entry of the modified myoferlin-containing nanoparticle of the present invention to the recipient cells of a subject within about 1 minutes, about 2 minutes, about 5 minutes, about 10 minutes, about 20 minutes, about 30 minutes, about 45 minutes, about 1 hour, about 2 hours, about 3 hours, about 4 hours, about 5 hours, about 6 hours, about 12 hours, about 18 hours, about 24 hours, about 36 hours, or about 48 hours of administration of the modified myoferlin-containing nanoparticle of the present invention to a subject. In an embodiment, more than about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, or about 100% of the payload is released from modified myoferlin-containing nanoparticle of the present invention within about 1 minutes, about 2 minutes, about 5 minutes, about 10 minutes, about 20 minutes, about 30 minutes, about 45 minutes, about 1 hour, about 2 hours, about 3 hours, about 4 hours, about 5 hours, about 6 hours, about 12 hours, about 18 hours, about 24 hours, about 36 hours, or about 48 hours of entry of the modified myoferlin-containing nanoparticle of the present invention into recipient cells. In an embodiment, the modified myoferlin protein of the present invention enhances the uptake of the modified-myoferlin containing nanoparticle of a present invention by the recipient cells of a subject by more than about 10%, about 50%, about 100%, about 200%, about 300%, about 400% or about 500% within about 1 minute, about 2 minutes, about 5 minutes, about 10 minutes, about 20 minutes, about 30 minutes, about 45 minutes, about 1 hour, about 2 hours, about 3 hours, about 4 hours, about 5 hours, about 6 hours, about 12 hours, about 18 hours, about 24 hours, about 36 hours, or about 48 hours of administration of the modified myoferlin-containing nanoparticle of the present invention to a subject. In an embodiment, the modified myoferlin protein of the present invention enhances release of the payload from the modified myoferlin-containing nanoparticle in recipient cells of a subject by at least about 1%, about 2%, about 3%, about 4%, about 5%, about 6%, about 7%, about 8%, about 9%, about 10%, about 20%, about 30%, about 40%, about 50%, about 60%, about 70%, about 80%, about 90%, or about 100% within about 1 minutes, about 2 minutes, about 5 minutes, about 10 minutes, about 20 minutes, about 30 minutes, about 45 minutes, about 1 hour, about 2 hours, about 3 hours, about 4 hours, about 5 hours, about 6 hours, about 12 hours, about 18 hours, about 24 hours, about 36 hours, or about 48 hours of administration of the modified myoferlin-containing nanoparticle of the present invention to a subject.

In an embodiment, the modified myoferlin protein of the present invention enhances the release of payload at the targeted site. In an embodiment, the target site comprises the interior of a recipient cell, a target cell, a cell at a target site, or a combination thereof. In an embodiment, the target site is outside of a recipient cell, a target cell, a cell at a target site, or a combination thereof. In an embodiment, a recipient cell comprises any cell capable of receiving any embodiment of the modified myoferlin-encapsulating nanoparticle of the present invention into the cell whether in vitro or in vivo. In an embodiment, the recipient cell is within a subject, harvested from a subject or derived from a subject. In an embodiment, a recipient cell is any cell affected by, influenced by or related to a disease for which the payload of the present invention is capable of providing therapeutic effects. In an embodiment, the recipient cell comprises a cell in contact with the modified myoferlin-containing nanoparticle of the present invention. In an embodiment, the recipient cell comprises a target cell, a cell at a target site, or a combination thereof. In an embodiment, the payload is activated after its release at the target site. The activation of a payload comprises the contact or binding of the payload to its target. In an embodiment, the activation of a payload comprises regulation of the expression or activity of the target of the payload. In an embodiment, the target comprises a nucleic acid, a polynucleotide, an amino acid, a peptide, a protein, a metabolite, or any components located at the target site. In an embodiment where the payload is a nucleic acid or a polynucleotide, the activation of payload comprises the contacting of the payload to a target nucleotide that is complementary to the nucleotide sequence of the payload.

In an embodiment, the modified myoferlin-encapsulating nanoparticle of the present invention comprises an exosome encapsulating any embodiment of the modified myoferlin of the present invention and one or more cargo RNAs. In an embodiment, the exosome encapsulating any embodiment of the modified myoferlin and one or more cargo RNAs of the present invention further comprises a fusion protein wherein the fusion protein comprises an exosome associated transmembrane protein fused to a packaging protein, and wherein each of the one or more cargo RNAs comprises a package RNA and a packaging domain. In an embodiment, the packaging domain of the cargo RNA binds to the packaging protein of the fusion protein to form a fusion protein cargo RNA complex, wherein the fusion protein and cargo RNA complex and the modified myoferlin of the present invention are packaged into the exosome. In an embodiment, the packaging protein of the present invention is endogenous to a subject. In an embodiment, one of the one or more of the any embodiment of the cargo RNA comprises a package RNA distinct from the package RNA of another of the one or more of the any embodiment of the cargo RNA such that one exosome may comprise two or more distinct package RNA. In an embodiment, the packaging protein comprises Ula protein. In an embodiment, the packaging domain comprises a UR domain.

In an embodiment, the cargo RNA comprises messenger RNAs, non-coding RNAs, or a combination thereof, wherein the non-coding RNA comprises long non-coding RNAs, circular RNAs, long-hairpin RNA, antisense RNAs, siRNAs, shRNAs, miRNAs, guide RNAs, or a combination thereof. In an embodiment, the cargo RNA comprises the packaging domain in the 3′-end or in the 5′-end of the package RNA. In an embodiment, the exosome associated transmembrane protein comprises CD63, CD9, CD81, PTGFRN or a combination thereof.

In an embodiment, the fusion protein of the present invention is in the form of a polynucleotide encoding said fusion protein such as but not limited to a fusion protein encoding plasmid. In an embodiment, the fusion protein comprises CD63-Ula protein. In an embodiment, nucleotide sequence of the CD63-Ula fusion protein encoding plasmid of the present invention is at least about 80%, about 85%, about 90%, about 95% or about 100% identical to SEQ ID NO. 28. In an embodiment, the amino the acid sequence of the CD63-Ula fusion protein is at least about 80%, about 85%, about 90%, about 95% or about 100% identical or similar to SEQ ID NO. 29. In an embodiment, the fusion protein comprises CD81-Ula protein. In an embodiment, nucleotide sequence of the CD81-Ula fusion protein encoding plasmid of the present invention is at least about 80%, about 85%, about 90%, about 95% or about 100% identical to SEQ ID NO. 30. In an embodiment, the amino acid sequence of the CD81-Ula fusion protein is at least about 80%, about 85%, about 90%, about 95% or about 100% identical or similar to SEQ ID NO. 31. In an embodiment, the fusion protein comprises PTGFRN-Ula protein. In an embodiment, nucleotide sequence of the PTGFRN-Ula fusion protein encoding plasmid of the present invention is at least about 80%, about 85%, about 90%, about 95% or about 100% identical to SEQ ID NO. 32. In an embodiment, the amino acid sequence of the PTGFRN-Ula fusion protein is at least about 80%, about 85%, about 90%, about 95% or about 100% identical or similar to SEQ ID NO. 33.

SEQ ID NO. 28 (nt CD63-U1a):
ATGGCGGTGGAAGGAGGAATGAAATGTGTGAAGTTCTTGCTCTACGTCCT
CCTGCTGGCCTTTTGCGCCTGTGCAGTGGGACTGATTGCCGTGGGTGTCG
GGGCACAGCTTGTCCTGAGTCAGACCATAATCCAGGGGGCTACCCCTGGC
TCTCTGTTGCCAGTGGTCATCATCGCAGTGGGTGTCTTCCTCTTCCTGGT
GGCTTTTGTGGGCTGCTGCGGGGCCTGCAAGGAGAACTATTGTCTTATGA
TCACGTTTGCCATCTTTCTGTCTCTTATCATGTTGGTGGAGGTGGCCGCA
GCCATTGCTGGCTATGTGTTTAGAGATAAGGTGATGTCAGAGTTTAATAA
CAACTTCCGGCAGCAGATGGAGAATTACCCGAAAAACAACCACACTGCTT
CGATCCTGGACAGGATGCAGGCAGATTTTAAGTGCTGTGGGGCTGCTAAC
TACACAGATTGGGAGAAAATCCCTTCCATGTCGAAGAACCGAGTCCCCGA
CTCCTGCTGCATTAATGTTACTGTGGGCTGTGGGATTAATTTCAACGAGA
AGGCGATCCATAAGGAGGGCTGTGTGGAGAAGATTGGGGGCTGGCTGAGG
AAAAATGTGCTGGTGGTAGCTGCAGCAGCCCTTGGAATTGCTTTTGTCGA
GGTTTTGGGAATTGTCTTTGCCTGCTGCCTCGTGAAGAGTATCAGAAGTG
GCTACGAGGTGATGgaattcggcggaggcgggtccATGGCAGTTCCCGAG
ACCCGCCCTAACCACACTATTTATATCAACAACCTCAATGAGAAGATCAA
GAAGGATGAGCTAAAAAAGTCCCTGTACGCCATCTTCTCCCAGTTTGGCC
AGATCCTGGATATCCTGGTATCACGGAGCCTGAAGATGAGGGGCCAGGCC
TTTGTCATCTTCAAGGAGGTCAGCAGCGCCACCAACGCCCTGCGCTCCAT
GCAGGGTTTCCCTTTCTATGACAAACCTATGCGTATCCAGTATGCCAAGA
CCGACTCAGATATCATTGCCAAGATGAAA

Uppercase sequence denotes CD63, lower case is linker sequence, and bold sequence is the U1 small nuclear ribonucleoprotein polypeptide A (U1 snRNP A).

SEQ ID NO. 29 (aa CD63-U1a):
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVFRDKVMSEFNNNFRQQMENYPKNNHTASILDRMQADFKCCGAAN
YTDWEKIPSMSKNRVPDSCCINVTVGCGINFNEKAIHKEGCVEKIGGWLR
KNVLVVAAAALGIAFVEVLGIVFACCLVKSIRSGYEVMefggggsMAVPE
TRPNHTIYINNLNEKIKKDELKKSLYAIFSQFGQILDILVSRSLKMRGQA
FVIFKEVSSATNALRSMQGFPFYDKPMRIQYAKTDSDIIAKMK

Uppercase sequence denotes CD63, lower case is linker sequence, and bold sequence is the U1 small nuclear ribonucleoprotein polypeptide A (U1 snRNP A).

SEQ ID NO. 30 (nt CD81-U1a):
ATGTCCGGACTCAGATCTCGAGCTCAAGCTTCCGGAGTGGAGGGCTGCACCAAG
TGCATCAAGTACCTGCTCTTCGTCTTCAATTTCGTCTTCTGGCTGGCTGGAGGCGT
GATCCTGGGTGTGGCCCTGTGGCTCCGCCATGACCCGCAGACCACCAACCTCCTG
TATCTGGAGCTGGGAGACAAGCCCGCGCCCAACACCTTCTATGTAGGCATCTACA
TCCTCATCGCTGTGGGCGCTGTCATGATGTTCGTTGGCTTCCTGGGCTGCTACGGG
GCCATCCAGGAATCCCAGTGCCTGCTGGGGACGTTCTTCACCTGCCTGGTCATCC
TGTTTGCCTGTGAGGTGGCCGCCGGCATCTGGGGCTTTGTCAACAAGGACCAGAT
CGCCAAGGATGTGAAGCAGTTCTATGACCAGGCCCTACAGCAGGCCGTGGTGGA
TGATGACGCCAACAACGCCAAGGCTGTGGTGAAGACCTTCCACGAGACGCTTGA
CTGCTGTGGCTCCAGCACACTGACTGCTTTGACCACCTCAGTGCTCAAGAACAAT
TTGTGTCCCTCGGGCAGCAACATCATCAGCAACCTCTTCAAGGAGGACTGCCACC
AGAAGATCGATGACCTCTTCTCCGGGAAGCTGTACCTCATCGGCATTGCTGCCAT
CGTGGTCGCTGTGATCATGATCTTCGAGATGATCCTGAGCATGGTGCTGTGCTGT
GGCATCCGGAACAGCTCCGTGTACGAATTCGGCGGAGGCGGGTCCATGGCAGTT
CCCGAGACCCGCCCTAACCACACTATTTATATCAACAACCTCAATGAGAAGATCA
AGAAGGATGAGCTAAAAAAGTCCCTGTACGCCATCTTCTCCCAGTTTGGCCAGAT
CCTGGATATCCTGGTATCACGGAGCCTGAAGATGAGGGGCCAGGCCTTTGTCATC
TTCAAGGAGGTCAGCAGCGCCACCAACGCCCTGCGCTCCATGCAGGGTTTCCCTT
TCTATGACAAACCTATGCGTATCCAGTATGCCAAGACCGACTCAGATATCATTGC
CAAGATGAAATAA
SEQ ID NO. 31 (aa CD81-U1a):
MSGLRSRAQASGVEGCTKCIKYLLFVFNFVFWLAGGVILGVALWLRHDPQTTNLLY
LELGDKPAPNTFYVGIYILIAVGAVMMFVGFLGCYGAIQESQCLLGTFFTCLVILFAC
EVAAGIWGFVNKDQIAKDVKQFYDQALQQAVVDDDANNAKAVVKTFHETLDCCG
SSTLTALTTSVLKNNLCPSGSNIISNLFKEDCHQKIDDLFSGKLYLIGIAAIVVAVIMIFE
MILSMVLCCGIRNSSVYEFGGGGSMAVPETRPNHTIYINNLNEKIKKDELKKSLYAIF
SQFGQILDILVSRSLKMRGQAFVIFKEVSSATNALRSMQGFPFYDKPMRIQYAKTDSD
IIAKMK
SEQ ID NO. 32 (nt PTGFRN-U1a):
ATGGGGCGCCTGGCCTCCAGGCCGCTGCTGCTGGCGCTCCTGTCGTTGGCTCTTT
GCCGAGGGCGTGTGGTGAGAGTCCCCACAGCGACCCTGGTTCGAGTGGTGGGCA
CTGAGCTGGTCATCCCCTGCAACGTCAGTGACTATGATGGCCCCAGCGAGCAAA
ACTTTGACTGGAGCTTCTCATCTTTGGGGAGCAGCTTTGTGGAGCTTGCAAGCAC
CTGGGAGGTGGGGTTCCCAGCCCAGCTGTACCAGGAGCGGCTGCAGAGGGGCGA
GATCCTGTTAAGGCGGACTGCCAACGACGCCGTGGAGCTCCACATAAAGAACGT
CCAGCCTTCAGACCAAGGCCACTACAAATGTTCAACCCCCAGCACAGATGCCAC
TGTCCAGGGAAACTATGAGGACACAGTGCAGGTTAAAGTGCTGGCCGACTCCCT
GCACGTGGGCCCCAGCGCGCGGCCCCCGCCGAGCCTGAGCCTGCGGGAGGGGGA
GCCCTTCGAGCTGCGCTGCACCGCCGCCTCCGCCTCGCCGCTGCACACGCACCTG
GCGCTGCTGTGGGAGGTGCACCGCGGCCCGGCCAGGCGGAGCGTCCTCGCCCTG
ACCCACGAGGGCAGGTTCCACCCGGGCCTGGGGTACGAGCAGCGCTACCACAGT
GGGGACGTGCGCCTCGACACCGTGGGCAGCGACGCCTACCGCCTCTCAGTGTCC
CGGGCTCTGTCTGCCGACCAGGGCTCCTACAGGTGTATCGTCAGCGAGTGGATCG
CCGAGCAGGGCAACTGGCAGGAAATCCAAGAAAAGGCCGTGGAAGTTGCCACC
GTGGTGATCCAGCCATCAGTTCTGCGAGCAGCTGTGCCCAAGAATGTGTCTGTGG
CTGAAGGAAAGGAACTGGACCTGACCTGTAACATCACAACAGACCGAGCCGATG
ACGTCCGGCCCGAGGTGACGTGGTCCTTCAGCAGGATGCCTGACAGCACCCTAC
CTGGCTCCCGCGTGTTGGCGCGGCTTGACCGTGATTCCCTGGTGCACAGCTCGCC
TCATGTTGCTTTGAGTCATGTGGATGCACGCTCCTACCATTTACTGGTTCGGGATG
TTAGCAAAGAAAACTCTGGCTACTATTACTGCCACGTGTCCCTGTGGGCACCCGG
ACACAACAGGAGCTGGCACAAAGTGGCAGAGGCCGTGTCTTCCCCAGCTGGTGT
GGGTGTGACCTGGCTAGAACCAGACTACCAGGTGTACCTGAATGCTTCCAAGGTC
CCCGGGTTTGCGGATGACCCCACAGAGCTGGCATGCCGGGTGGTGGACACGAAG
AGTGGGGAGGCGAATGTCCGATTCACGGTTTCGTGGTACTACAGGATGAACCGG
CGCAGCGACAATGTGGTGACCAGCGAGCTGCTTGCAGTCATGGACGGGGACTGG
ACGCTAAAATATGGAGAGAGGAGCAAGCAGCGGGCCCAGGATGGAGACTTTATT
TTTTCTAAGGAACATACAGACACGTTCAATTTCCGGATCCAAAGGACTACAGAGG
AAGACAGAGGCAATTATTACTGTGTTGTGTCTGCCTGGACCAAACAGCGGAACA
ACAGCTGGGTGAAAAGCAAGGATGTCTTCTCCAAGCCTGTTAACATATTTTGGGC
ATTAGAAGATTCCGTGCTTGTGGTGAAGGCGAGGCAGCCAAAGCCTTTCTTTGCT
GCCGGAAATACATTTGAGATGACTTGCAAAGTATCTTCCAAGAATATTAAGTCGC
CACGCTACTCTGTTCTCATCATGGCTGAGAAGCCTGTCGGCGACCTCTCCAGTCC
CAATGAAACGAAGTACATCATCTCTCTGGACCAGGATTCTGTGGTGAAGCTGGA
GAATTGGACAGATGCATCACGGGTGGATGGCGTTGTTTTAGAAAAAGTGCAGGA
GGATGAGTTCCGCTATCGAATGTACCAGACTCAGGTCTCAGACGCAGGGCTGTAC
CGCTGCATGGTGACAGCCTGGTCTCCTGTCAGGGGCAGCCTTTGGCGAGAAGCA
GCAACCAGTCTCTCCAATCCTATTGAGATAGACTTCCAAACCTCAGGTCCTATAT
TTAATGCTTCTGTGCATTCAGACACACCATCAGTAATTCGGGGAGATCTGATCAA
ATTGTTCTGTATCATCACTGTCGAGGGAGCAGCACTGGATCCAGATGACATGGCC
TTTGATGTGTCCTGGTTTGCGGTGCACTCTTTTGGCCTGGACAAGGCTCCTGTGCT
CCTGTCTTCCCTGGATCGGAAGGGCATCGTGACCACCTCCCGGAGGGACTGGAA
GAGCGACCTCAGCCTGGAGCGCGTGAGTGTGCTGGAATTCTTGCTGCAAGTGCAT
GGCTCCGAGGACCAGGACTTTGGCAACTACTACTGTTCCGTGACTCCATGGGTGA
AGTCACCAACAGGTTCCTGGCAGAAGGAGGCAGAGATCCACTCCAAGCCCGTTT
TTATAACTGTGAAGATGGATGTGCTGAACGCCTTCAAGTATCCCTTGCTGATCGG
CGTCGGTCTGTCCACGGTCATCGGGCTCCTGTCCTGTCTCATCGGGTACTGCAGCT
CCCACTGGTGTTGTAAGAAGGAGGTTCAGGAGACACGGCGCGAGCGCCGCAGGC
TCATGTCGATGGAGATGGACGAATTCGGCGGAGGCGGGTCCATGGCAGTTCCCG
AGACCCGCCCTAACCACACTATTTATATCAACAACCTCAATGAGAAGATCAAGA
AGGATGAGCTAAAAAAGTCCCTGTACGCCATCTTCTCCCAGTTTGGCCAGATCCT
GGATATCCTGGTATCACGGAGCCTGAAGATGAGGGGCCAGGCCTTTGTCATCTTC
AAGGAGGTCAGCAGCGCCACCAACGCCCTGCGCTCCATGCAGGGTTTCCCTTTCT
ATGACAAACCTATGCGTATCCAGTATGCCAAGACCGACTCAGATATCATTGCCAA
GATGAAATAG
SEQ ID NO. 33 (aa PTGFRN-U1a):
MGRLASRPLLLALLSLALCRGRVVRVPTATLVRVVGTELVIPCNVSDYDGPSEQNFD
WSFSSLGSSFVELASTWEVGFPAQLYQERLQRGEILLRRTANDAVELHIKNVQPSDQ
GHYKCSTPSTDATVQGNYEDTVQVKVLADSLHVGPSARPPPSLSLREGEPFELRCTA
ASASPLHTHLALLWEVHRGPARRSVLALTHEGRFHPGLGYEQRYHSGDVRLDTVGS
DAYRLSVSRALSADQGSYRCIVSEWIAEQGNWQEIQEKAVEVATVVIQPSVLRAAVP
KNVSVAEGKELDLTCNITTDRADDVRPEVTWSFSRMPDSTLPGSRVLARLDRDSLVH
SSPHVALSHVDARSYHLLVRDVSKENSGYYYCHVSLWAPGHNRSWHKVAEAVSSP
AGVGVTWLEPDYQVYLNASKVPGFADDPTELACRVVDTKSGEANVRFTVSWYYR
MNRRSDNVVTSELLAVMDGDWTLKYGERSKQRAQDGDFIFSKEHTDTFNFRIQRTT
EEDRGNYYCVVSAWTKQRNNSWVKSKDVFSKPVNIFWALEDSVLVVKARQPKPFF
AAGNTFEMTCKVSSKNIKSPRYSVLIMAEKPVGDLSSPNETKYIISLDQDSVVKLENW
TDASRVDGVVLEKVQEDEFRYRMYQTQVSDAGLYRCMVTAWSPVRGSLWREAAT
SLSNPIEIDFQTSGPIFNASVHSDTPSVIRGDLIKLFCIITVEGAALDPDDMAFDVSWFA
VHSFGLDKAPVLLSSLDRKGIVTTSRRDWKSDLSLERVSVLEFLLQVHGSEDQDFGN
YYCSVTPWVKSPTGSWQKEAEIHSKPVFITVKMDVLNAFKYPLLIGVGLSTVIGLLSC
LIGYCSSHWCCKKEVQETRRERRRLMSMEMDEFGGGGSMAVPETRPNHTIYINNLN
EKIKKDELKKSLYAIFSQFGQILDILVSRSLKMRGQAFVIFKEVSSATNALRSMQGFPF
YDKPMRIQYAKTDSDIIAKMK

In an embodiment, nucleotide sequence of the UR domain is at least about 80%, about 85%, about 90%, about 95% or about 100% identical to SEQ ID NO. 34 or SEQ ID NO. 35. In an embodiment, the cargo RNA further comprises stabilizing domain comprising the OH domain and/or the MorrisMotif domain wherein the nucleotide sequence of the OH domain is at least about 80%, about 85%, about 90%, about 95% or about 100% identical to SEQ ID NO. 36 and the nucleotide sequence of the MorrisMotif domain is at least about 80%, about 85%, about 90%, about 95% or about 100% identical to SEQ ID NO. 37. In an embodiment, the packaging domain of the present invention comprises 1 to 10 repeats of the UR domain.

SEQ ID NO. 34: AATCCATTGCACTCCGGATT
SEQ ID NO. 35: AATCCATTGCACTCCGGATTT
SEQ ID NO. 36: CTGCAGATATCCAGCACAGTGGC
SEQ ID NO. 37: GCGCAGCGCGCGCAGCGC

In an embodiment, the cargo RNA of the of the present invention further comprises a SINE-derived nuclear RNA LOcalizatIoN (SIRLOIN) sequence. In an embodiment, SIRLOIN sequence is upstream of the packaging domain of the cargo RNA. In an embodiment, SIRLOIN sequence is upstream of the UR sequence of the cargo RNA. In an embodiment, the nucleotide sequence of the SIRLOIN sequence is at least about 80%, about 85%, about 90%, about 95% or about 100% identical to SEQ ID NO. 38.

SEQ ID NO. 38:
CGCCTCCCGGGTTCAAGCGATTCTCCTGCCTCAGCCTCCCGA

It is to be understood that both the foregoing general description and detailed description are exemplary and explanatory only and are not restrictive of the invention, as claimed. In general, the terms used in the disclosure should not be construed to limit the technology to the specific embodiments disclosed in the specification, unless the above detailed description explicitly defines such terms. Accordingly, the actual scope of the technology encompasses the disclosed embodiments and all equivalent ways of practicing or implementing the technology.

### Examples

### Materials and Methods

### Cell Culture

HEK293 cells were cultured at 37° C./5% CO₂ in n Dulbecco's Modified Eagle's Medium (ThermoFisher) supplemented with 10% Fetal Bovine Serum and 1% Penicillin, Streptomycin, Glutamine. The day before transfection, 100,000 cells (producers) were plated in each of eight wells of a 12 well plate, and 30,000 cells (receivers) in each of eight well inserts fitted for 12-well plates (Costar® 12 mm Transwell®, 0.4 μm Pore Polyester Membrane Inserts, STEMCELL Technologies). Lipofectamine 2000 transfection reagent was used in a 1:1.3 of μg of plasmid DNA to μL of transfection reagent. The plasmid combinations used in this experiment are shown in table 1. The medium was replaced with fresh medium after 6 hours and the inserts swells were moved on top of the transfected producer cells. After 48 hours, the receiver cells were trypsinised, collected, and pelleted in 1.5 Eppendorf tubes.

TABLE 1
Combinations of plasmids used in this experiment. 500 ng of
pDB30 were used in each well to standardize CD63-luciferase
expression. The masses of all other plasmids were calculated
based on their mass to ensure equal amount of plasmid copies
in each well. All plasmids were being stored at −20° C.
in TE buffer before transfection.

| Sample | plasmids | |
|---|---|---|
| 1 | Untransfected negative control | |
| 2 | pDB30 (500 ng) | pcDNA3.1 (410 ng) |
| 3 | pDB30 (500 ng) | WT MYOF (886 ng) |
| 4 | pDB30 (500 ng) | C2A MYOF (476 ng) |
| 5 | pDB30 (500 ng) | C2A-C2B MYOF (528 ng) |
| 6 | pDB30 (500 ng) | C2A-DysF-FerA MYOF (551 ng) |
| 7 | pDB30 (500 ng) | C2F-C2G MYOF (531 ng) |
| 8 | pDB30 (500 ng) | pDB68 (Cx43) (491 ng) |

### Luciferase Assay

Luciferase assay was performed with Nano-Glo® Luciferase Assay System (Promega). Nano-Glo® Luciferase Assay Buffer reagent and Nano-Glo® Luciferase Assay Substrate were thawed at room temperature and mixed in a 50:1 ratio. Each cell pellet was resuspended in 150 μL of PBS and 150 μL of luciferase buffer were added to the cell lysates. Luminescence readings were performed in three technical replicates by adding 100 μL of each lysate to three wells of a Greiner F-bottom 96-well plate. The reading was performed using FLUOstar OPTIMA (BMG LABTECH).

### Effects of MYOF on EV Uptake Relative to the EXOtic System

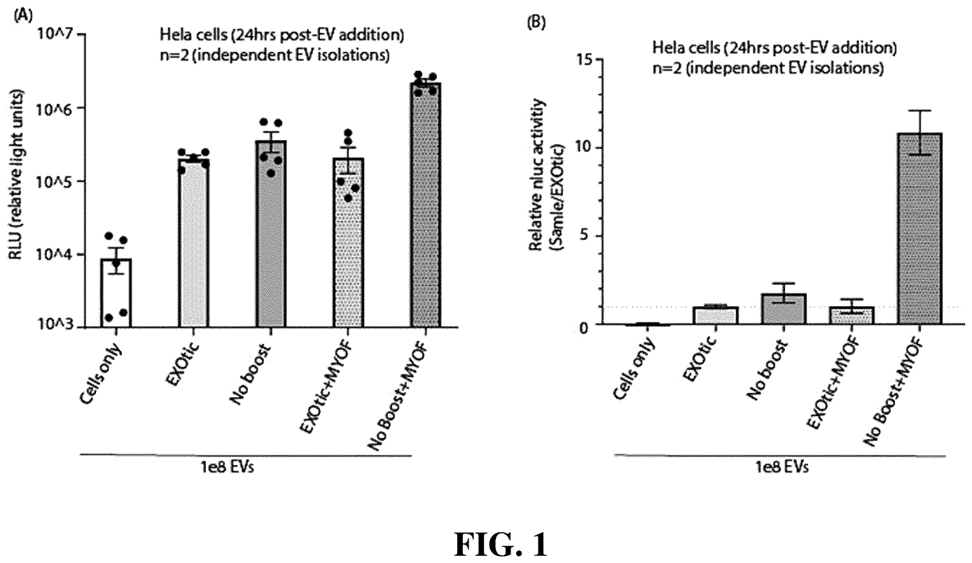

MYOF has been reported to be packaged into extracellular vesicles (EVs) and may facilitate release of EV payloads into recipient cells (1). To determine if MYOF is functional in enhanced release of EV payloads into recipient cells we generated nanoluciferase (nLuc) packaged EVs using the EXOtic system with EXOtic booster components, such as Connexion 43 (CX43) (2) or with full-length MYOF. Transfected HEK293 cell generated EVs were produced and assessed for whether the booster plasmid was necessary for EV production, and if MYOF instead of CX43 increased EV uptake in HeLa cells (FIG. 1A). When standardized to the control of standard EXOtic treated cells, the presence of full-length myoferlin substantially increased the uptake of nanoluciferase (nLuc) into the recipient cells (FIG. 1B). These data suggest that the presence of full-length MYOF in EVs functionally increases EV uptake and release of payloads in target cells.

### Myoferlin and Truncated Variants Enhance Transfer of EV Packaged Nanoluciferase.

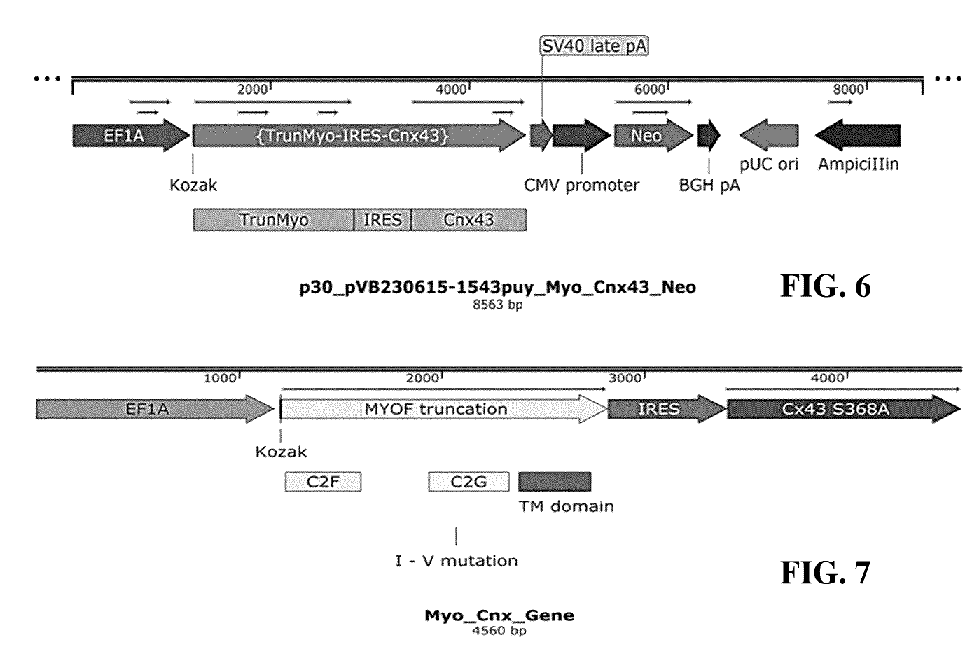

MYOF is a multi-domain protein that is involved in receptor recycling and endocytosis. The C2-domains can bind to lipid layers and change the structure of lipid packing, forming regional distorted membrane structure responsible for fusion and fission events (FIG. 2A-E). Next, to determine if there is a domain that facilitates the observed enhanced release features of MYOF in EVs, various truncated forms of MYOF were developed (FIGS. 2B-E). Notably, the C2A, C2B, Fer and DysFN domains were retained in the designs, as these are well studied regions that have been shown to be involved in Calcium dependent trafficking and membrane repair. The transmembrane domain was also retained in each construct, as this is critical for protein binding to the lipid membrane and fusiogenic events and a histone tag was embedded downstream for protein analysis and protein purification (FIGS. 2B-E). To determine how well these various MYOF truncations facilitate extracellular vesicle (EV) mediated receptor cell uptake, we screened transfected HEK293 cells for transwell transfer of EV packaged nLuc. Both pC2A-Dys-Fer and pC2F-C2G significantly enhanced transfer of nLuc relative to EXOtic CX43 and full-length Myoferlin (FIG. 3). These data outline 2 unexpected outcomes from the use of particular truncated segments of MYFO in the transfer of EV nanoparticles to recipient cells, those are (1) that the C2F and C2G and transmembrane spanning elements are all that is required for cellular uptake of EV packaged payloads and (2) that this relatively short, ~2.5 kb sequence in pC2F-C2G MYOF (FIG. 3) can profoundly affect fusogenic uptake of nLuc. This relatively short sequence allows for this approach to be amendable to both cellular and synthetic nanosystems and could find utility in cell and gene therapies and synthetic lipid nanoparticles (LNP) to enhance both uptake and endosomal release of nucleic acid or protein payloads. Towards this goal we have developed a dual protein expressing endosomal release system (FIGS. 6 and 7) that can be used to greatly enhance the efficacy and delivery of RNA and nucleic acid payloads.

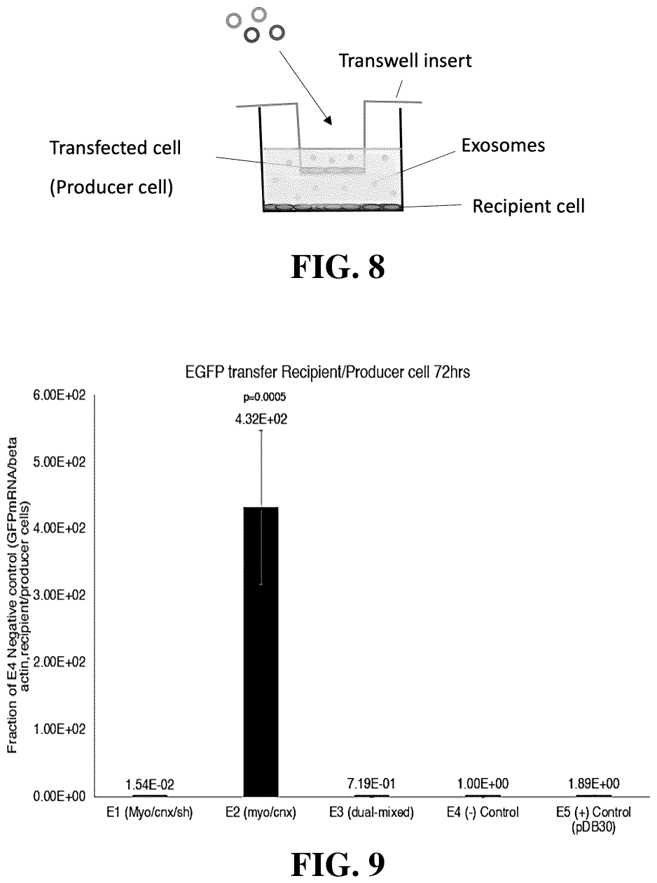

We set out to determine the optimal enhancer construct for generating efficient RNA packaged EVs that can transfect recipient cells. To do this we used transwell assays (FIG. 8) and transfected HEK293 cells with plasmids (Table 2) and grow in transwell (0.4 μm) plate culture with recipient HEK293 cells. In Table 2, the p18 plasmid encodes GFP protein, CD domain, UR domain, and hydromycin resistance gene. The p25 plasmid encodes CD63 protein and L7ae protein. The p29 plasmid encodes a truncated myoferlin protein (C2F-C2G-TM), a connexin 43 S368A protein, and shRNAs targeting VPS4B/CHMP. The p30 plasmid encodes a truncated myoferlin protein consisting of C2F-C2G-TM domains, a connexin 43 S368A protein, and neomycin resistance gene. The p30 plasmid is shown in further detail in FIG. 6. The p55 plasmid is a pUC19 plasmid that does not encode any truncated myoferlin. The p54 plasmid encodes the connexin 43 S368A protein.

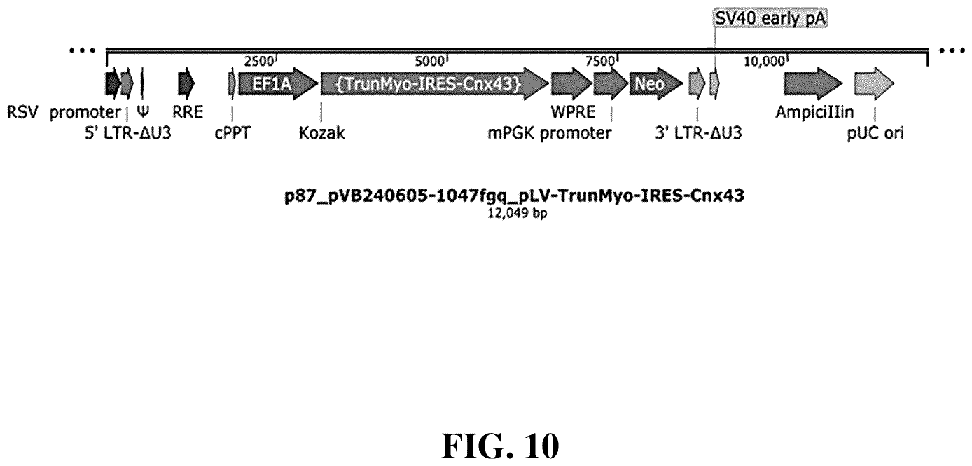

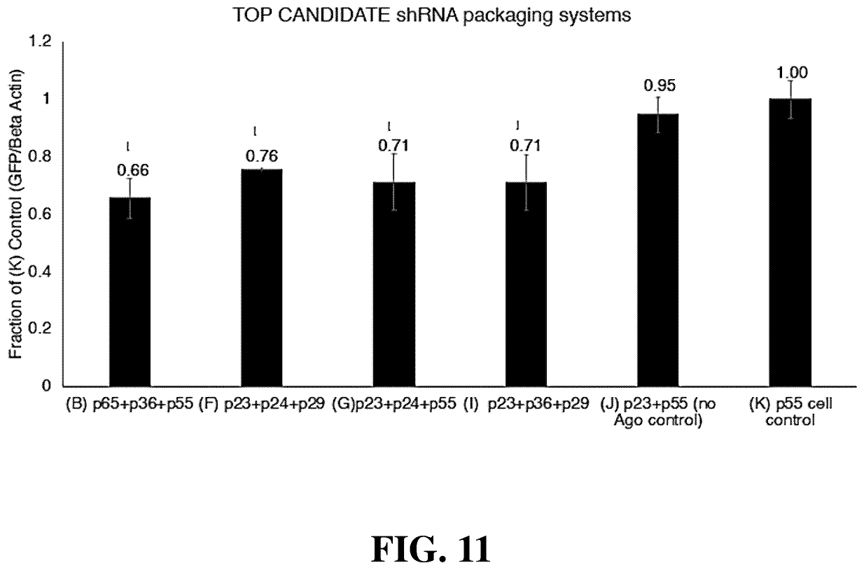

Results summarized in FIG. 9 show that the optimal enhancer construct is p30_pVB230615-1543puy_Myo_Cnx43_Neo, which contains the DNA sequence expressing the recombinant enhancer protein (SEQ ID NO. 27) and expresses the resulting protein (SEQ ID NO. 25 and SEQ ID NO. 26). Notably, we have transferred p30_pVB230615-1543puy_Myo_Cnx43_Neo to a lentiviral backbone for stable integration and generation of cell lines and find this new plasmid p87_pVB240605-1047fgq_pLV-TrunMyo-IRES-Cnx43 (FIG. 10) was found to work as well as p30_pVB230615-1543puy_Myo_Cnx43_Neo and can be generated as a lentiviral vector with Neomycin selection. These data suggest that the enhancer system, p87_pVB240605-1047fgq_pLV-TrunMyo-IRES-Cnx43 and/or p30_pVB230615-1543puy_Myo_Cnx43_Neo are functional and the optimal constructs for generating exosomes which can deliver therapeutic mRNAs and long antisense non-coding RNAs to target cells.

TABLE 2
Plasmids transfect into HEK cells to
determine the optimal enhancer construct.

| | Plasmids number in bold, plasmids to | DNA to |
|---|---|---|
| | transfect (ratio of plasmid to cells; | add for |
| Treatment | 1 μg/10\hat{ }6 cells) | 10\hat{ }4 cells |
| (1) 1-Enhancer | -(a) p18 pGFP-CD-UR-Hygro_VB230706- | 25 ng |
| | 1681mwk | 25 ng |
| | -(b) p25 pCD63-L7ae_VB230613-1717fme | 50 ng |
| | -(c) p29 pMyo-cnx43-shVp>Chmp_ | |
| | pVB230726-1193mwk | |
| (2) 2-Enhancer | -(a) p18 pGFP-CD-UR-Hygro_VB230706- | 25 ng |
| | 1681mwk | 25 ng |
| | -(b) p25 pCD63-L7ae_VB230613-1717fme | 50 ng |
| | -(c) p30 pVB230615- | |
| | 1543puy_Myo_Cnx43_Neo | |
| (3) 3-Enhancer | -(a) p18 pGFP-CD-UR-Hygro_VB230706- | 25 ng |
| | 1681mwk | 25 ng |
| | -(b) p25 pCD63-L7ae_VB230613-1717fme | 25 ng |
| | -(c) p30 pVB230615- | 25 ng |
| | 1543puy_Myo_Cnx43_Neo | |
| | -(d) p29 pMyo-cnx43- | |
| | shVp>Chmp_pVB230726-1193mwk | |
| (4) 4-Enhancer | -(a) p18 pGFP-CD-UR-Hygro_VB230706- | 25 ng |
| (−) control | 1681mwk | 25 ng |
| | -(b) p25 pCD63-L7ae_VB230613-1717fme | 50 ng |
| | -(c) p55 puc19 (KMW2) | |
| (5) 5-Enhancer | -(a) p18 pGFP-CD-UR-Hygro_VB230706- | 25 ng |
| (+) control | 1681mwk | |
| | -(b) p25 pCD63-L7ae_VB230613-1717fme | 25 ng |
| | -(c) p54 pDB68 (Conx43) | 50 ng |

While insightful for longer forms of RNAs, mRNAs, antisense RNAs, etc, the effect of this enhancer system on small hairpin RNA (shRNA) delivery recipient cells was not known. To determine to what extent the enhancer affects shRNA delivery, we screened several HEK producer cells using the transwell system (FIG. 8) transfected with various plasmid combinations containing the Ago2 packaging construct from U.S. provisional patent application No. 63/598,499, shRNAs targeted to GFP, the reporter transgene expressed in the recipient cells, and various enhancer constructs, including one P29=pRP-Neo-shVPS4B/shCHMP4C-EF1A>{TrunMyo-IRES-Cnx43} which expresses shRNAs targeted to CHMP4C and VPS4B, found to enhance exosome numbers being produced in the producer cells. Notably, we make 2 key observations regarding the enhancer system and shRNA delivery using the Ago2 packaging system from U.S. provisional patent application No. 63/598,499. Based on the work presented in FIG. 9, we observe that the addition of the truncated myoferlin with Cnx43, expressed from the context of P29=pRP-Neo-shVPS4B/shCHMP4C-EF1A>{TrunMyo-IRES-Cnx43} did not appreciably affect shRNA efficacy of transfer compared to no enhancer (FIG. 9, Table 3, compared to no enhance control (G) p23+p24+p55). Secondly, the over-expression of shVPS4B and shCHMP4C does not affect exosomes delivered shRNA efficacy, suggesting that larger numbers of exosomes can be generated from cells containing shRNAs. Collectively, these data demonstrate that the truncated myo-Cnx43 enhancer system can be used with shRNAs, synergistically with shVPS4B/shCHMP4C.

TABLE 3
Plasmid combinations used in FIG. 6. The various plasmid combinations were
generated and found to functionally transfer shRNAs to target cells

| Vector combination | shGFP | shRNA packer | EV Enhancer |
|---|---|---|---|
| (B) p65 + p36 + p55 | (+)p65=PE17- | (+) p36={Ago2-IRES- | (−) p55=pUC19 |
| | Ago2+Ago-shGFPb | Cnx43} | |
| (F) p23 + p24 + p29 | (+) p23=pRP-{U6- | (+) p24=pRP-Bsd- | (+) P29=pRP-Neo- |
| | shGFP}-CMV>Puro | EF1A>{Ago-2} | shVPS4B/shCHMP4C- |
| | | | EF1A>{TrunMyo-IRES- |
| | | | Cnx43} |
| (G)p23 + p24 + p55 | (+) p23=pRP-{U6- | (+) p24=pRP-Bsd- | (−) p55=pUC19 |
| | shGFP}-CMV>Puro | EF1A>{Ago-2} | |
| (I) p23 + p36 + p29 | (+) p23=pRP-{U6- | (+) p36={Ago2-IRES- | (+) P29=pRP-Neo- |
| | shGFP}-CMV>Puro | Cnx43} | shVPS4B/shCHMP4C- |
| | | | EF1A>{TrunMyo-IRES- |
| | | | Cnx43} |

In summary we report here the functional utility of the TrunMyo-Cnx43 enhancer system and how this system can be used with U.S. provisional patent application No. 63/598,499 to generate highly functional RNA based gene regulatory exosomes and that this system solves one of the issues plaquing exosome and nanoparticle delivery, which is delivery beyond the endosome.

It can be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

References which are hereby each incorporated in each of their entirety
1. Blomme A, Fahmy K, Peulen O, Costanza B, Fontaine M, Struman I, et al. Myoferlin is a novel exosomal protein and functional regulator of cancer-derived exosomes. Oncotarget. 2016; 7 (50): 83669-83.
2. Kojima R, Bojar D, Rizzi G, Hamri GC, El-Baba MD, Saxena P, et al. Designer exosomes produced by implanted cells intracerebrally deliver therapeutic cargo for Parkinson's disease treatment. Nat Commun. 2018; 9 (1): 1305.

1. A modified myoferlin protein comprising one or more C2 domains and one or more non-C2 domains
wherein the one or more C2 domains comprises a C2A domain, a C2B domain, a C2C domain, a C2D domain, a C2E domain, a C2F domain, a C2G domain, or a combination thereof;
wherein the one or more non-C2 domain comprise a transmembrane domain, a FerA domain, a FerB domain, a DysFN-1, domain, a DysFN-2 domain, or a combination thereof; and
wherein the modified myoferlin protein does not comprise one or more of C2B domain, C2C domain, C2D domain and/or C2E domains.

2. The modified myoferlin protein of claim 1, wherein the modified myoferlin protein comprises a polypeptide comprising a C2F domain, a C2G domain, a transmembrane domain, or a combination thereof.

3. The modified myoferlin protein of claim 1, wherein the modified myoferlin protein comprises a polypeptide consisting of a C2F domain, a C2G domain, a transmembrane domain

4. The modified myoferlin protein of claim 2, wherein the C2G domain comprises an I-V mutation from the C2G domain of a wildtype protein.

5. The modified myoferlin protein of claim 2, wherein the amino acid sequence of the C2F domain is at least about 80%, about 85%, about 90%, about 95%, or about 100% identical or similar to SEQ ID NO. 1, the amino acid sequence of the C2G domain is at least about 80%, about 85%, about 90%, about 95%, or about 100% identical or similar to SEQ ID NO. 2, and the amino acid sequence of the transmembrane domain is at least about 80%, about 85%, about 90%, about 95%, or about 100% identical or similar to SEQ ID NO. 3.

6. The modified myoferlin protein of claim 5, wherein the amino acid sequence of the modified myoferlin protein is at least about 80%, about 85%, about 90%, about 95%, or about 100% identical or similar to SEQ ID NO. 25.

7. The modified myoferlin protein of claim 1, wherein the modified myoferlin protein is encoded by a modified myoferlin protein encoding polynucleotide that encodes a C2F domain, a C2G domain, a transmembrane domain, or a combination thereof.

8. The modified myoferlin protein of claim 7, wherein the C2G domain comprises an I-V mutation from the C2G domain of a wildtype protein.

9. The modified myoferlin protein of claim 7, wherein the modified myoferlin protein encoding polynucleotide comprises a first nucleotide sequence, a second nucleotide sequence and a third nucleotide sequence wherein nucleotide sequence of the first nucleotide sequence is at least about 80%, about 85%, about 90%, about 95%, or about 100% identical to SEQ ID NO. 13, nucleotide sequence of the second nucleotide sequence is at least about 80%, about 85%, about 90%, about 95%, or about 100% identical to SEQ ID NO. 14, and nucleotide sequence of the third nucleotide sequence at least about 80%, about 85%, about 90%, about 95%, or about 100% identical to SEQ ID NO. 15.

10. The modified myoferlin protein of claim 1, wherein the modified myoferlin protein comprises a polypeptide comprising a C2A domain, a FerA domain, a FerB domain, a DysFN-1 domain, a DysFN-2 domain, a transmembrane domain, or a combination thereof.

11. The modified myoferlin protein of claim 1, wherein the modified myoferlin protein comprises a polypeptide consisting of a C2A domain, a FerA domain, a FerB domain, a DysFN-1 domain, a DysFN-2 domain, a transmembrane domain, or a combination thereof.

12. The modified myoferlin protein of claim 11, wherein amino acid sequence of the C2A domain is at least about 80%, about 85%, about 90%, about 95%, or about 100% identical or similar to SEQ ID NO. 4, amino acid sequence of the FerA domain is at least about 80%, about 85%, about 90%, about 95%, or about 100% identical or similar to SEQ ID NO. 5, amino acid sequence of the FerB domain is at least about 80%, about 85%, about 90%, about 95%, or about 100% identical or similar to SEQ ID NO. 6, amino acid sequence of the DysFN-1 domain is at least about 80%, about 85%, about 90%, about 95%, or about 100% identical or similar to SEQ ID NO. 7, amino acid sequence of the DysFN-2 domain is at least about 80%, about 85%, about 90%, about 95%, or about 100% identical or similar to SEQ ID NO. 8, and amino acid sequence of the transmembrane domain is at least about 80%, about 85%, about 90%, about 95%, or about 100% identical or similar to SEQ ID NO. 3.

13. The modified myoferlin protein of claim 11, wherein the modified myoferlin protein is encoded by a modified myoferlin protein encoding polynucleotide comprising a first nucleotide sequence, a second nucleotide sequence, a third nucleotide sequence, a fourth nucleotide sequence, a fifth nucleotide sequence and a sixth nucleotide sequence wherein nucleotide sequence of the first nucleotide sequence is at least about 80%, about 85%, about 90%, about 95%, or about 100% identical to SEQ ID NO. 16, nucleotide sequence of the second nucleotide sequence is at least about 80%, about 85%, about 90%, about 95%, or about 100% identical to SEQ ID NO. 17, nucleotide sequence of the third nucleotide sequence is at least about 80%, about 85%, about 90%, about 95%, or about 100% identical to SEQ ID NO. 18, nucleotide sequence of the fourth nucleotide sequence is at least about 80%, about 85%, about 90%, about 95%, or about 100% identical to SEQ ID NO. 19, nucleotide sequence of the fifth nucleotide sequence is at least about 80%, about 85%, about 90%, about 95%, or about 100% identical to SEQ ID NO. 20, and nucleotide sequence of the sixth nucleotide sequence is at least about 80%, about 85%, about 90%, about 95%, or about 100% identical to SEQ ID NO. 15.

14. The modified myoferlin protein of claim 1, further comprising a connexin 43 protein wherein the modified myoferlin protein is fused with the connexin 43 protein.

15. The modified myoferlin protein of claim 14, wherein the connexin 43 protein comprises a S368A mutation.

16. The modified myoferlin protein of claim 15, wherein amino acid sequence of the connexin 43 is at least about 80%, about 85%, about 90%, about 95%, or about 100% identical or similar to SEQ ID NO. 26.

17. The modified myoferlin protein of claim 14, wherein the modified myoferlin protein fused with the connexin 43 protein comprises a polypeptide comprising a first amino acid sequence and a second amino acid sequence wherein amino acid sequence of the first amino acid sequence is at least about 80%, about 85%, about 90%, about 95%, or about 100% identical or similar to SEQ ID NO. 25 and amino acid sequence of the second amino acid sequence is at least about 80%, about 85%, about 90%, about 95%, or about 100% identical or similar to SEQ ID NO. 26.

18. The modified myoferlin protein of claim 14, wherein the modified myoferlin protein fused with the connexin 43 protein is encoded by a dual protein polynucleotide wherein nucleotide sequence of the dual protein polynucleotide is at least about 80%, about 85%, about 90%, about 95%, or about 100% identical to SEQ ID NO. 27.

19. The composition of claim 1, further comprises a nanoparticle and a payload, wherein the nanoparticle encapsulates the modified myoferlin protein and the payload.

20. The composition of claim 19, wherein the nanoparticle comprises liposome, lipid nanoparticle, polymer nanoparticle, lipid-polymer hybrid nanoparticle, virus-like particle, exosome or a combination thereof.

21. The composition of claim 19, wherein the nanoparticle further comprises one or more conjugates for targeted delivery to a recipient cell in a subject.

22. The composition of claim 19, wherein the payload comprises a therapeutic agent, a prophylactic agent, a diagnostic agent, a nutritional agent, or a combination thereof.

23. The composition of claim 19, wherein the payload comprises a polynucleotide, a polypeptide, a recombinant protein, a small-molecule drug, or a combination thereof.
